(19) United States
(12) Patent Application Publication
(10) Pub. No.: US 2023/0392117 A1
(43) Pub. Date: Dec. 7, 2023

(54) COMPOSITIONS AND METHODS FOR REGULATING T CELLS

(71) Applicant: UNIVERSITY OF PITTSBURGH-OF THE COMMONWEALTH SYSTEM OF HIGHER EDUCATION, Pittsburgh, PA (US)

(72) Inventors: Craig Alan BYERSDORFER, Cheswick, PA (US); Erica BRAVERMAN, Pittsburgh, PA (US)

(21) Appl. No.: 18/034,246

(22) PCT Filed: Apr. 30, 2021
(86) PCT No.: PCT/US2021/030175

§ 371 Date: Apr. 27, 2023

Related U.S. Application Data
(60) Provisional application No. 63/107,621, filed on Oct. 30, 2020.

(51) Int. Cl. C12N 5/0783; C12N 9/12; C12N 15/86; A61K 39/00

(52) U.S. Cl. CPC C12N 5/0636; C12N 9/12; C12Y 207/11031; C12N 15/86; A61K 39/4611; C12N 2501/2302; A61K 39/464402; A61K 39/4631; A61K 39/464412; C12N 2510/00; C12N 2740/15043; A61K 39/464462

(57) ABSTRACT

Provided herein are methods of making a T cell comprising increasing a level of a AMPKγ2 polypeptide in the T cell wherein the T cell has the increased level of the AMPKγ2 polypeptide has an increased oxidative metabolism as compared to the control. The T cells made using these methods are included herein and can be used for increasing an immune response in a subject.

### CROSS-REFERENCE TO RELATED APPLICATIONS

This application claims the benefit of U.S. Provisional Application No. 63/107,621, filed Oct. 30, 2020, which is expressly incorporated herein by reference in its entirety.

### STATEMENT REGARDING FEDERALLY SPONSORED RESEARCH OR DEVELOPMENT

This invention was made with government support under grant number W81XWH-19-1-0291 awarded by the Department of Defense. The government has certain rights in this invention.

### FIELD

The present disclosure relates to methods for engineering T cells and uses of the engineered T cells for regulating immune responses.

### BACKGROUND

Approaches which use primary immune cells to target antigens expressed predominantly on cancer cells have the potential to improve cancer control and provide long-term cures. However, full translation of this game changing technology is hindered by an inability of in vitro activated T cells to survive well in vivo. Similarly, transfer of autologous T cells bearing chimeric antigen receptors (CARs) has revolutionized the treatment of certain types of cancers. However, a significant subset of patients receiving CAR T cells continue to suffer relapse or incomplete responses due to a lack of CAR T cell persistence. Increasing the in vivo persistence of tumor-reactive lymphocytes can improve tumor clearance. What is needed is a method to improve T cell persistence in vivo. The compositions and methods disclosed herein address these and other needs.

### SUMMARY

Provided herein are methods of making a T cell comprising increasing a level of a AMPKγ2 polypeptide in the T cell as compared to a control, wherein the AMPKγ2 polypeptide comprises SEQ ID NO:1, and wherein the T cell having the increased level of the AMPKγ2 polypeptide has an increased oxidative metabolism as compared to the control. In some embodiments, the AMPKγ2 polypeptide consists of a sequence at least 95% identical to SEQ ID NO:1.

In some aspects, the method comprises introducing a vector into the T cell, wherein the vector comprises a polynucleotide sequence encoding the AMPKγ2 polypeptide. The polynucleotide sequence can be at least 90% identical to SEQ ID NO: 2 in some embodiments. In some embodiments, the vector is a lentiviral vector.

In some aspects, the method further comprises culturing the T cell in the presence of IL-2. The T cell can be cultured for any amount of time, but in some embodiments, is cultured for about 2 to 12 days.

The methods provided herein result in T cells having an increased level of an AMPKγ2 polypeptide have one or more characteristics as compared to a control T cell, those characteristics being selected from the group consisting of: 1) an increased oxidative metabolism, 2) less differentiated, 3) an increased extracellular acidification rate (ECAR), 4) an increased level of proliferation, 5) an increased in vivo survival time, and 6) an increased effector function. In some aspects, the T cell can be a CD4⁺ T cell or a CD8⁺ T cell. The T cell can be a CAR T cell, an effector T cell, an effector memory T cell, a T_{EMRA}, a central memory T cell, an effector T regulatory cell (Treg), an effector memory Treg, or a T memory stem cell (T_{SCM}). In some embodiments, T cell is an effector Treg or an effector memory Treg and has an increased suppressor function as compared to the control. In some embodiments, the T cell is a tumor infiltrating T cell (or TIL), or a T cell that is obtained from a tumor.

Also included herein is a method of increasing an immune response in a subject comprising administering to the subject a therapeutically effective amount of a T cell having an increased level of an AMPKγ2 polypeptide as compared to a control, wherein the AMPKγ2 polypeptide comprises SEQ ID NO:1. In some aspects, the AMPKγ2 polypeptide comprises a sequence at least 95% identical to SEQ ID NO: 1. In some aspects, the T cell comprises a vector and the vector comprises a polynucleotide sequence encoding the AMPKγ2 polypeptide. The polynucleotide sequence can be at least 90% identical to SEQ ID NO: 2 in some aspects. The method can further include culturing the T cell in the presence of IL-2 prior to administration, in some aspects, for about 2 to 12 days. In some embodiments, the administered T cell having the increased level of the AMPKγ2 polypeptide has one or more characteristics as compared to a control T cell, those characteristics being selected from the group consisting of: 1) an increased oxidative metabolism, 2) less differentiated, 3) an increased extracellular acidification rate (ECAR), 4) an increased level of proliferation, 5) an increased in vivo survival time, and 6) an increased effector function. In some aspects the administered T cell is a CD4⁺ T cell. In some aspects, the administered T cell is a CD8⁺ T cell. In some aspects, the administered T cell is a CAR T cell, an effector T cell, an effector memory T cell, a T_{EMRA}, a central memory T cell, an effector T regulatory cell (Treg), an effector memory Treg, or a T memory stem cell (T_{SCM}). In some embodiments, the T cell is a tumor infiltrating T cell (or TIL), or a T cell that is obtained from a tumor.

Further included herein is a method of making a population of T cells comprising increasing a level of a AMPKγ2 polypeptide in the T cells as compared to a control T cell population, wherein the AMPKγ2 polypeptide comprises SEQ ID NO:1, and wherein the population of T cells has an increased amount of CD4+ T cells as compared to the control T cell population.

### DESCRIPTION OF DRAWINGS

FIG. 1 is a schematic showing a lentiviral construct for expression of AMPKγ2 and a control construct.

FIGS. 2(A-B) show transduction of T cells with lentiviral constructs (A) increases AMPKγ2 expression at the RNA as shown with RT-PCR analysis and (B) protein levels as shown with Western Blotting.

FIGS. 3(A-B) show expression of GFP and AMPKγ2 in primary human T cells. (A) Shows that primary human T cells were stimulated with CD3/CD28 Dynabeads and then transduced with AMPKγ2, empty vector, or mock transduced. GFP was measured by flow cytometry on day 7 post-transduction. (B) Shows that, in a separate experiment, 10e5 GFP+ T cells were flow-sorted into 10% TCA, cell lysates precipitated and run on immunoblot probing for AMPKγ2. Lamin B1 served as a loading control. Immunoblots are representative of 2 experiments performed in triplicate. E=Empty vector.

FIGS. 4(A-B) show increased AMPK signaling in AMPKγ2-transduced cells. (A) Shows that T cells were transduced with AMPKγ2 or empty vector and levels of phosphorylated AMPK (*P-AMPK) or *P-ULK1 were measured in GFP+ sorted cells by immunoblot. (B) Shows that blots were scanned and densitometry measurements performed using ImageJ software. In each case, the level of the phosphorylated protein was compared to the total levels. ****=p<0.0001, **=p<0.01.

FIG. 5 shows increased growth in AMPKγ2-transduced T cells in standard culture conditions.

FIG. 6 shows increased CD4+ T cell percentages in AMPKγ2-transduced T cell populations. T cells were mock transduced or transduced with empty vector or AMPKγ2 lentiviral constructs and evaluated for CD4 versus CD8 expression on day 7 post-transduction by flow cytometry. Results are representative of every experiment run (n>6). In addition, a similar increase in CD4+ T cells is also seen at day 10, day 14, and upon re-stimulation.

FIGS. 7(A-B) show improved growth in AMPKγ2-transduced cells. T cells were transduced with AMPKγ2 or empty vector and cell numbers measured on day 3, followed by expansion in IL-2. Cell numbers were then generated on days 5, 7, and 10 post-transduction and compared as a fold-change back to the numbers on day 3. CD8 had an initial lag in growth followed by subsequent expansion. In the end (e.g. day 10), both CD4+(FIG. 7A) and CD8⁺ T (FIG. 7B) cells increased in number to a greater degree when transduced with AMPKγ2.

FIGS. 8(A-B) show that AMPKγ2-transduced T cells are less differentiated. T cells were transduced with AMPKγ2 or empty vector and evaluated on day +7 (FIG. 8A) or day +14 (FIG. 8B) post-transduction for differentiation status based on CD62L versus CD45RA or CD62L versus CD27 expression. In each case, less differentiated cells reside in the upper right hand quadrant (e.g. CD62L+CD45RA+). Results are consistent with those obtained in a second, independent experiment.

FIGS. 9(A-B) show that AMPKγ2-transduced T cells increase mitochondrial membrane potential and mitochondrial mass. Primary human T cells were transduced with AMPKγ2 (orange) or empty vector (blue) and mitochondrial membrane potential (FIG. 9A) or mitochondrial mass (FIG. 9B) were measured by flow cytometry on day 5 post-transduction.

FIGS. 10(A-B) show increased respiratory capacity in AMPKγ2-transduced cells. T cells were transduced with AMPKγ2 or empty vector and sorted for GFP+ cells on day 7 post-transduction. (A) Two days later, cells were placed on a Seahorse metabolic analyzer and their oxygen consumption (OCR) was measured. (B) T cells were re-simulated with CD3CD28 Dynabeads for 24 hours prior to analysis on the Seahorse machine. ****=p<0.0001, **=p<0.01, n.s.=not significant.

FIG. 11 shows dual expression of AMPKγ2 and a CAR. Jurkat T cells were mock transduced or transduced with the CAR construct alone (blue fluorescent protein (BFP)) or with the CAR construct+AMPKγ2 (BFP/GFP double positive). On day 7 post-transduction, T cells were analyzed for expression of one or both proteins by flow cytometry. Data are consistent with results from two independent experiments.

FIG. 12 shows a schematic depicting the design of a co-culture experiment.

FIGS. 13(A-B) show increased baseline oxidation and increased spare respiratory capacity in AMPKγ2-transduced T cells following CAR stimulation. Jurkat T cells were dual-transduced with CAR and AMPKγ2 constructs and placed in a Seahorse metabolic analyzer to measure 02 consumption rates (OCR). In FIG. 13A, cells were placed in nutrient limited media for 6 hours prior to analysis, while in FIG. 13B, cells were co-cultured with CD19-bearing target cells for 24 hours prior to analysis. Co-culture with CAR target-bearing cells increases the spare respiratory capacity (SRC), suggestive of increased reserves of oxidative metabolism in these cells.

FIGS. 14(A-C) show increased proliferation in AMPKγ2-transduced cells. Primary human T cells were transduced with AMPKγ2 or empty lentiviral vector and the number of GFP+ cells was measured over time. (A) shows doubling time of GFP+ cells was assessed between days 5 and 7 in four separate donor samples, with normalization of each Empty sample set to a value of 100. (B) Cell cycle analysis was performed on transduced cells on day 9 in culture using flow cytometric analysis. (C) Fewer AMPKγ2 cells were in the GO/1 phase in both CD4 and CD8 T cells, with results compiled from 3 separate donors. *p<0.05, **p<0.01, ****p<0.0001.

FIGS. 15(A-B) show increased mTOR signaling in AMPKγ2-transduced cells. (A) Primary human T cells were transduced with AMPKγ2 or empty lentiviral vector and the MFI of intracellular phosphorylated-S6 (PS6) or 4EBP1 was quantitated on day 9 in culture. (B) P-S6 and P-4EBP1 levels were analyzed in transduced T cells from four separate donors (with Empty set to a relative value of 100 in each case). **p<0.01, ***p<0.001.

FIG. 16 shows AMPKγ2-transduced T cells remain less-differentiated. AMPKγ2-transduced T cells retained higher levels of both CD62L and CCR7 during in vitro culture, indicative of a less-differentiated phenotype. Results are from day 9 T cells.

FIGS. 17(A-D) show elevated PD-1 (A) and CD25 (B) expression but equivalent LAG3 (C) TIM3 (D) levels in AMPKγ2-transduced cells. Human T cells were transduced with AMPKγ2 or empty lentiviral vector and expanded in IL-2. On day 10 in culture, cells were evaluated for expression of the activation markers PD-1 (A) and CD25 (B) and exhaustion markers LAG3 (C) and TIM3 (D). All graphs are composites of primary human T cells from 3 to 4 individual donors. *p<0.05, **p<0.01.

FIGS. 18(A-B) show equivalent cytokine production in AMPKγ2-transduced cells. Human T cells were transduced with AMPKγ2 or empty lentiviral vector and expanded in IL-2. On day 9 in culture, T cells were stimulated with PMA and ionomycin in the presence of monensin for six hours, followed by intracellular cytokine detection. Representative FACs analysis results for CD8 T cells are shown in (A), with data compiled from 3 separate donors (B).

FIGS. 19(A-G) show increased 02 consumption in AMPKγ2-transduced cells. AMPKγ2- and Empty-transduced T cells were expanded in IL-2 until day 9, then analyzed on a Seahorse Metabolic Analyzer for (A) oxygen consumption rates (OCR) and (B) Extracellular Acidification Rates. (C-D) In an independent experiment, day 9 AMPKγ2- versus Empty-transduced T cells were stimulated (C) or not (D) overnight with CD3/CD28 beads prior to Seahorse analysis. (E-F) Basal OCR, spare respiratory capacity (SRC), and maximal OCR in resting (E) or stimulated (F) transduced T cells from three independent donors on day 9 of culture. (G) Baseline ECAR values from either resting (top) or stimulated (bottom) T cells from three independent donors. Values are normalized to a relative value of 100 in Empty controls. *p<0.05, **p<0.01, ****p<0.0001 by unpaired Student T test.

### DETAILED DESCRIPTION

Disclosed herein are methods and compositions for making a T cell comprising increasing a level of an AMPKγ2 polypeptide in the T cell as compared to a control T cell. In some embodiments, the T cell is a regulatory T cell. Also disclosed herein are methods of increasing an immune response in a subject comprising administering to the subject a therapeutically effective amount of a T cell having an increased level of an AMPKγ2 polypeptide as compared to a control T cell. In some aspects, the AMPKγ2 polypeptide comprises SEQ ID NO: 1, SEQ ID NO: 3, or a fragment thereof. The methods include transducing a vector into the T cell, wherein the vector (e.g., a lentiviral vector) comprises a polynucleotide sequence encoding an AMPKγ2 polypeptide, and wherein the polynucleotide comprises SEQ ID NO: 2, SEQ ID NO: 4, or a fragment thereof. The method has shown to be surprisingly effective at creating T cells that have increased growth in vitro, an increased CD4/CD8 ratio, increased in vivo persistence, are less differentiated, and/or have increased levels of oxidative metabolism as compared to a control.

Terms used throughout this application are to be construed with ordinary and typical meaning to those of ordinary skill in the art. However, Applicant desires that the following terms be given the particular definition as provided below.

### Terminology

As used in the specification and claims, the singular form “a,” “an,” and “the” include plural references unless the context clearly dictates otherwise. For example, the term “a cell” includes a plurality of cells, including mixtures thereof.

The term “about” as used herein when referring to a measurable value such as an amount, a percentage, and the like, is meant to encompass variations of 20%, +10%, +5%, or +1% from the measurable value.

“Activate”, “activating”, and “activation” mean to increase an activity, response, condition, or other biological parameter. This may also include, for example, a 10% increase in the activity, response, “or condition, as compared to the native or control level. Thus, the increase can be a 10, 20, 30, 40, 50, 60, 70, 80, 90, 100%, or any amount of reduction in between as compared to native or control levels.

“Administration” to a subject includes any route of introducing or delivering to a subject an agent (e.g., a T cell). Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purposes. A control can be “positive” or “negative.” In some embodiments, the control described herein refers to a T cell that has not been transduced with an AMPKγ2 polynucleotide.

“Differentiation” refers to the process by which immature and unspecialized cells mature and take on specialized forms and/or functions. Differentiation state can be determined based on a cell's expression profile. In some embodiments, a less differentiated T cell is CD62L⁺CD45RA⁺ or CD62LCD27⁺.

The term “extracellular acidification rate” or “ECAR” refers to a measurement of a cell's rate of glycolysis, and is predominantly or wholly a measure of lactic acid secretion per unit time.

As used herein, “effector function” refers to one or more of 1) secretion of cytotoxins such as perforin and granzymes, 2) target cell killing, 3) secretion of macrophage activating cytokines such as IFN-γ, GM-CSF and TNF-α, and 4) secretion of B cell activating cytokines such as IL-4, IL-5, and IL-10. In some embodiments, an increase in effector function in a CD8⁺ T cell includes in an increase in either or both secretion of cytotoxins and target cell killing. In some embodiments, an increase in effector function in a CD4+ T cell includes an increase in secretion of macrophage activating cytokines. In some embodiments, an increase in effector function in a CD4+ T cell includes an increase in secretion of B cell activating cytokines.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription of DNA and translation of mRNA results in the protein.

“Expression vector” or “vector” comprises a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.)

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property of the sequence from which it is derived such as increasing in vivo persistence, decreasing differentiation, and/or increasing levels of oxidative metabolism in a T cell in which the fragment is expressed.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

As used herein, the term “expression” refers to either or both “gene expression” and “protein expression.” “Gene expression” refers to the process by which polynucleotides are transcribed into mRNA and “protein expression” refers to the process by which mRNA is translated into peptides, polypeptides, or proteins. If the polynucleotide is derived from genomic DNA, expression may include splicing of the mRNA in a eukaryotic cell. “Gene overexpression” refers to the overproduction of the mRNA transcribed from the gene, at a level that is at least about 2.5 times higher, at least about 5 times higher, or at least about 10 times higher than the expression level detected in a control sample. “Protein overexpression” includes the overproduction of the protein product encoded by a gene at a level that is at least about 2.5 times higher, at least about 5 times higher, or at least about 10 times higher than the expression level detected in a control sample.

“Inhibit”, “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “in vivo persistence” refers to a cell's survival time and/or effector function in vivo. Using the methods or the compositions herein, the in vivo persistence of a T cell is at least about 1.5 times, at least about 2 times, at least about 3 times, at least about 4 times, at least about 5 times, at least about 10 times, at least about 15 times, at least about 20 times, at least about 30 times, at least about 40 times, at least about 50 times, at least about 60 times, at least about 80 times, at least about 100 times, at least about 500 times, at least about 1000 times higher than a control T cell. In some embodiments, a T cell's survival time is at least about 1.5 times, at least about 2 times, at least about 3 times, at least about 4 times, at least about 5 times, at least about 10 times, at least about 15 times, at least about 20 times, at least about 30 times, at least about 40 times, at least about 50 times, at least about 60 times, at least about 80 times, at least about 100 times, at least about 500 times, at least about 1000 times higher than a control T cell. In some embodiments, a T cell's effector function is at least about 1.5 times, at least about 2 times, at least about 3 times, at least about 4 times, at least about 5 times, at least about 10 times, at least about 15 times, at least about 20 times, at least about 30 times, at least about 40 times, at least about 50 times, at least about 60 times, at least about 80 times, at least about 100 times, at least about 500 times, at least about 1000 times higher than a control T cell.

The term “nucleic acid” as used herein means a polymer composed of nucleotides, e.g. deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides. (Used together with “polynucleotide” and “polypeptide”.) Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, “operatively linked” can indicate that the regulatory sequences useful for expression of the coding sequences of a nucleic acid are placed in the nucleic acid molecule in the appropriate positions relative to the coding sequence so as to effect expression of the coding sequence. This same definition is sometimes applied to the arrangement of coding sequences and/or transcription control elements (e.g. promoters, enhancers, and termination elements), and/or selectable markers in an expression vector. The term “operatively linked” can also refer to the arrangement of polypeptide segments within a single polypeptide chain, where the individual polypeptide segments can be, without limitation, a protein, fragments thereof, linking peptides, and/or signal peptides. The term operatively linked can refer to direct fusion of different individual polypeptides within the single polypeptides or fragments thereof where there are no intervening amino acids between the different segments as well as when the individual polypeptides are connected to one another via one or more intervening amino acids.

The term “oxidative metabolism” refers to the chemical process in which oxygen is used to make energy from carbohydrates. A cell's oxidative metabolism level can be determined by any method known to those of skill in the art. In some embodiments, the level of oxidative metabolism is equivalent to the oxidative consumption rate (OCR), which can be determined using, for example, a Seahorse Extracellular Flux Analyzer.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, P A, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

The term “promoter” as used herein is defined as a DNA sequence recognized by the synthetic machinery of the cell, or introduced synthetic machinery, required to initiate the specific transcription of a polynucleotide sequence.

As used herein, the term “regulatory sequence” means a nucleic acid sequence which is required for expression of a gene product operably linked to the regulatory sequence. In some instances, this sequence may be the core promoter sequence and in other instances, this sequence may also include an enhancer sequence and other regulatory elements which are required for expression of the gene product. The regulatory sequence may, for example, be one which expresses the gene product in a tissue specific manner.

“Recombinant” used in reference to a gene refers herein to a sequence of nucleic acids that are not naturally occurring. The non-naturally occurring sequence may include a recombination, substitution, deletion, or addition of one or more bases with respect to the nucleic acid sequence originally present in the natural genome.

The terms “identity” “identical to” and “homology” shall be construed to mean the percentage of nucleotide bases or amino acid residues in the candidate sequence that are identical with the bases or residues of a corresponding sequence to which it is compared, after aligning the sequences and introducing gaps, if necessary to achieve the maximum percent identity for the entire sequence, and not considering any conservative substitutions as part of the sequence identity. Neither N- nor C-terminal extensions nor insertions shall be construed as reducing identity or homology. A polynucleotide or polynucleotide region (or a polypeptide or polypeptide region) that has a certain percentage (for example, 80%, 85%, 90%, or 95%) of “sequence identity” to another sequence means that, when aligned over their full lengths, that percentage of bases (or amino acids) are the same in comparing the two sequences. This alignment and the percent homology or sequence identity can be determined using software programs known in the art. In one embodiment, default parameters are used for alignment. In one embodiment a BLAST program is used with default parameters. In one embodiment, BLAST programs BLASTN and BLASTP are used with the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “suppress”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

“Suppressor function” refers herein to a T cell's suppression of the activation, proliferation or cytokine production of other T cells, B cells and/or dendritic cells. A regulatory T cell, or a Treg, has suppressor function, and the present invention can be used to increase this suppressor function or to increase the number of Treg cells in a T cell population.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of a disease (e.g., a cancer).

“Therapeutically effective amount” or “therapeutically effective dose” of a composition (e.g. a composition comprising an agent) refers to an amount that is effective to achieve a desired therapeutic result. In some embodiments, a desired therapeutic result is a mitigation of a cancer. In some embodiments, a desired therapeutic result is an increase in a T cell driven immune response. In some embodiments, a desired therapeutic result is an increase in a regulatory T cell (TReg) immune response. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as mitigation of a cancer. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The word “vector” refers to any vehicle that carries a polynucleotide into a cell for the expression of the polynucleotide in the cell. The vector may be, for example, a plasmid, a phage particle, or a nanoparticle. Once transformed into a suitable host cell, the vector may replicate and function independently of the host genome, or may in some instances, integrate into the genome itself. In some embodiments, the vector is a DNA construct containing a DNA sequence which is operably linked to a suitable control sequence capable of effecting the expression of the DNA in a suitable host cell. Such control sequences can include a promoter to effect transcription, an optional operator sequence to control such transcription, a sequence encoding suitable mRNA ribosome binding sites, and sequences which control the termination of transcription and translation. In other embodiments, the vector is a lipid nanoparticle. Lipid nanoparticles can be used to deliver mRNA to a host cell for expression of the mRNA in the host cell.

### Compositions

Disclosed herein is a method of making a T cell comprising increasing a level of an AMPKγ2 polypeptide in the T cell as compared to a control. This method has been shown to be surprisingly effective at increasing T cell oxidative metabolism levels, increasing T cell in vivo persistence as compared to a control, increasing suppressive functionality in regulatory T cells, and/or increasing the amount or percentage of CD4+ T cells in a T cell population. In some embodiments, the level of the AMPKγ2 polypeptide is increased via transducing a vector into the T cell, wherein the vector comprises a polynucleotide sequence encoding the AMPKγ2 polypeptide. Accordingly, provided herein are compositions comprising AMPKγ2 encoding polynucleotides.

AMP-activated protein kinase (AMPK) is a heterotrimeric kinase complex composed of a catalytic α subunit with serine/threonine kinase activity, as well as β and γ subunits that regulate its activation and substrate specificity. AMPK activation is regulated by the binding of adenylate nucleotides (i.e., ATP, ADP and AMP) to the nucleotide-binding sites of the 7 subunit, which precedes activating phosphorylation events on the α and β subunits. There are three isoforms of the γ-subunits, isoforms 1, 2, and 3. “AMPKγ2” refers herein to a polypeptide that that synthesizes and hydrolyzes cyclic adenosine 5′-diphosphate-ribose, and in humans, is encoded by the PRKAG2 gene. In some embodiments, the AMPKγ2 polypeptide is that identified in one or more publicly available databases as follows: HGNC: 9386, Entrez Gene: 51422, Ensembl: ENSG00000106617, OMIM: 602743, and UniProtKB: Q9UGJ0. In some embodiments, the AMPKγ2 polypeptide comprises SEQ ID NO:1. In some embodiments, the AMPKγ2 polypeptide comprises SEQ ID NO: 3. In some embodiments, the AMPKγ2 polypeptide comprises a polypeptide sequence having at or greater than about 80%, about 85%, about 90%, about 95%, or about 98% homology with SEQ ID NO:1 or SEQ ID NO: 3, or a polypeptide comprising a portion of SEQ ID NO:1 or SEQ ID NO: 3. The AMPKγ2 polypeptide of SEQ ID NO:1 or SEQ ID NO: 3 may represent an immature or pre-processed form of mature AMPKγ2, and accordingly, included herein are mature or processed portions of the AMPKγ2 polypeptide in SEQ ID NO:1 and SEQ ID NO: 3. In some embodiments, the AMPKγ2 described herein is a full-length polypeptide of AMPKγ2 that comprises a polypeptide sequence at least about 80%, about 85%, about 90%, about 95%, about 97%, about 98%, about 99%, or about 99.5% identical to SEQ ID NO: 3. In some embodiments, the AMPKγ2 described herein is a truncated version of AMPKγ2 polypeptide that consists of a polypeptide sequence at least about 80%, about 85%, about 90%, about 95%, about 97%, about 98%, about 99%, or about 99.5% identical to SEQ ID NO: 1.

In some embodiments, the AMPKγ2 polypeptide is operably linked to a degradation motif. “Degradation motif” is used herein to refer to a polypeptide sequence that targets an operably linked amino acid sequence, e.g., an AMPKγ2 polypeptide, for degradation. In some embodiments, the degradation motif is an E3 ubiquitination motif. In some embodiments, the degradation motif is activated for targeting upon a change in the motif's conformation.

As discussed above, AMPK is a master regulator of metabolism, promoting oxidative phosphorylation when nutrients are scarce. It has been shown that expression of AMPK in a hepatic cell line can regulate cellular metabolism. However, whether increasing AMPK levels (e.g., AMPKγ2) in an immune cell (e.g., a T cell) remains unexplored. Notably, technical challenges still remain in the prior art for efficient transduction of AMPKγ2 polynucleotide into a T cell (e.g., primary T cells, T cell lines, or CAR T cells). The methods disclosed herein are surprisingly effective at transducing an AMPKγ2 polynucleotide into to a T cell to increase the level of AMPKγ2 polypeptide in the T cell.

Therefore, provided herein are vectors comprising an AMPKγ2 encoding polynucleotide sequence. In some embodiments, the polynucleotide sequence is at least about 80%, about 85%, about 90%, about 95%, about 97%, about 98%, about 99%, about 99.5% identical to SEQ ID NO: 2, SEQ ID NO: 4, or a fragment thereof. In some embodiments, the AMPKγ2 polynucleotide sequence encodes an AMPKγ2 polypeptide that comprises SEQ ID NO:1. In some embodiments, the AMPKγ2 polynucleotide sequence encodes an AMPKγ2 polypeptide that comprises SEQ ID NO: 3. In some embodiments, the AMPKγ2 polynucleotide sequence encodes an AMPKγ2 polypeptide that comprises a polypeptide sequence having at or greater than about 80%, about 85%, about 90%, about 95%, or about 98% homology with SEQ ID NO:1 or SEQ ID NO: 3, or a polypeptide comprising a portion of SEQ ID NO:1 or SEQ ID NO: 3. In some embodiments, the AMPKγ2 polynucleotide is operably linked to a polynucleotide sequence that encodes a degradation motif.

In some embodiments, the vector is a viral vector. “Viral vector” as disclosed herein means, in respect to a vehicle, any virus, virus-like particle, virion, viral particle, or pseudotyped virus that comprises a nucleic acid sequence that directs packaging of a nucleic acid sequence in the virus, virus-like particle, virion, viral particle, or pseudotyped virus. In some embodiments, the virus, virus-like particle, virion, viral particle, or pseudotyped virus is capable of transferring a vector (such as a nucleic acid vector) into and/or between host cells. In some embodiments, the virus, virus-like particle, virion, viral particle, or pseudotyped virus is capable of transferring a vector (such as a nucleic acid vector) into and/or between target cells, such as a hepatocyte in the liver of a subject. Importantly, in some embodiments, the virus, virus-like particle, virion, viral particle, or pseudotyped virus is capable of transporting into a nucleus of a target cell (e.g., a hepatocyte). The term “viral vector” is also meant to refer to those forms described more fully in U.S. Patent Application Publication U.S. 2018/0057839, which is incorporated herein by reference for all purposes. Suitable viral vectors include, e.g., adenoviruses (Mitani et al., Hum. Gene Ther. 5:941-948, 1994), adeno-associated virus (AAV) (Goodman et al., Blood 84:1492-1500, 1994), vaccinia viruses, herpesviruses, baculoviruses and retroviruses (Agrawal et al., Exper. Hematol. 24:738-747, 1996), parvoviruses, and lentiviruses (Naidini et al., Science 272:263-267, 1996). In some embodiments, the viral vector is a lentiviral vector.

In some embodiments, the AMPKγ2 coding polynucleotide sequence is operatively linked to a second polynucleotide sequence that encodes a ribosomal skipping sequence (or self-cleaving peptide). In some embodiments, the ribosomal skipping sequence is introduced between the AMPKγ2 polypeptide and a protein, wherein the protein can be located upstream of the N-terminus of the AMPKγ2 polypeptide or downstream of the C-terminus of the AMPKγ2 polypeptide. The ribosomal skipping sequence helps generates two proteins by having the ribosome fall off in between the two sequences. Accordingly, in some aspects, the vector comprises a ribosomal skipping sequence that is operatively linked to the AMPKγ2 coding polynucleotide sequence. In some embodiments, the ribosomal skipping sequence is T2A. In some embodiments, the T2A comprises a sequence at least about 80%, about 85%, about 90%, about 95%, about 97%, about 98%, about 99%, about 99.5% identical to SEQ ID NO: 5 or a fragment thereof.

In some embodiments, the vector further comprises additional promoter elements, e.g., enhancers that regulate the frequency of transcriptional initiation. Typically, these are located in the region 30-110 bp upstream of the start site of the nucleic acid sequence mentioned above (e.g., the nucleic acid sequence encoding AMPKγ2 polypeptide), although a number of promoters have recently been shown to contain functional elements downstream of the start site as well.

The spacing between promoter elements frequently is flexible, so that promoter function is preserved when elements are inverted or moved relative to one another. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1 alpha (EF-1α). In some embodiments, the EF-1α comprises a polynucleotide sequence at least about 80%, about 85%, about 90%, about 95%, about 97%, about 98%, about 99%, about 99.5% identical to SEQ ID NO: 8 or a fragment thereof. However, other promoter sequences may also be used, including, but not limited to the simian virus 40 (SV40), early promoter, mouse mammary tumor virus (MMTV), human immunodeficiency virus (HIV) long terminal repeat (LTR) promoter, MoMuLV promoter, PGK-1 promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter as well as synthetic protein, such as a CAG promoter. Further, the invention should not be limited to the use of constitutive promoters, inducible promoters are also contemplated as part of the invention. The use of an inducible promoter provides a molecular switch capable of turning on expression of the polynucleotide sequence which it is operatively linked when such expression is desired, or turning off the expression when expression is not desired.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (GFP) (e.g., Ui-Tei et al., 2000 FEBS Letters 479: 79-82). Suitable expression systems are well known and may be prepared using known techniques or obtained commercially. Accordingly, in some embodiments, vector of any preceding aspect further comprises a reporter gene. In some embodiments, the reporter gene is GFP. In one example, the GFP gene is in frame with the AMPKγ2 and/or the T2A sequence. If the ribosome reads through and does not “skip”, this makes one giant fusion protein. In some aspects, the reporter gene is also a suicide gene such as the RQR8 gene. In one example, the RQR8 gene is in frame with the AMPKγ2 and/or the T2A sequence.

In some embodiments, the GFP gene used herein comprises a sequence at least about 80%, about 85%, about 90%, about 95%, about 97%, about 98%, about 99%, about 99.5% identical to SEQ ID NO: 6 or a fragment thereof. In some embodiments, the RQR8 gene used herein comprises a sequence at least about 80%, about 85%, about 90%, about 95%, about 97%, about 98%, about 99%, about 99.5% identical to SEQ ID NO: 9 or a fragment thereof.

In some embodiments, the recombinant polynucleotide disclosed herein comprises a promoter, a polynucleotide sequence encoding AMPKγ2, a polynucleotide sequence encoding a T2A peptide, a GFP reporter gene or a RQR8 gene, and a linker sequence and/or a spacer sequence. In some embodiments, the vector comprises a sequence at least about 80%, about 85%, about 90%, about 95%, about 97%, about 98%, about 99%, about 99.5% identical to SEQ ID NO: 7, SEQ ID NO: 10, or a fragment thereof.

In some further embodiments, the AMPKγ2-containing vector described in any of the proceeding aspects further comprises a nucleic acid encoding a chimeric antigen receptor (CAR). The term “chimeric antigen receptor (CAR),” as used herein, may refer to artificial T-cell receptors, chimeric T-cell receptors, or chimeric immunoreceptors, for example, and encompass engineered receptors that graft an artificial specificity onto a particular immune effector cell (e.g., a T cell). In some embodiments, CARs comprise an intracellular domain, a transmembrane domain, and an extracellular domain comprising a tumor associated antigen binding region. Methods for making CAR are known in the art. See, e.g., U.S. Pat. No. 9,540,445 and International Patent Application Publication No. WO2014011987, both incorporated by reference herein in their entireties. As used herein, a T cell comprising a CAR is referred to as a “CAR T cell.”

Also provided herein are T cells comprising a AMPKγ2 encoding polynucleotide sequence. The AMPKγ2 encoding polynucleotide sequence can be any described herein. In some embodiments, the T cells comprise at least two vectors: a vector that includes an AMPKγ2 encoding polynucleotide sequence and a second vector that includes a polynucleotide sequence that encodes a CAR.

The T cell can be of any type, including, but not limited to, a CD4⁺ T cell or a CD8⁺ T cell. The CD4⁺ T cell can be a T helper cell (Th), a regulatory T cell (Treg) or a follicular T helper cell (Tfh). The CD4⁺ T helper T cell can be a Th1, a Th2, a Th9, a Th17, or a Th22. For example, Th1 releases IFN-7 and TNF; Th2 releases TL-4 (an important survival factor for B-type lymphocytes), IL-5 and IL-13; Th9 produces IL-9; Treg secretes IL-10 (a cytokine with an immunosuppressive function, maintaining expression of FOXP3 transcription factor needed for suppressive function of Treg on other cells) and TGF-β; and Th17 produces IL-17 (a cytokine playing an important role in host defense against bacteria, and fungi). In some aspects, the T cell is an effector T cell (CD25+, CD45RA+/−, CD45RO+/−, CD127−), an effector memory T cell (CD25−, CD45RA−, CD45RO+, CD127+), a T_{EMRA} (CD25−, CD45RA+, CD45RO+, CD127+), a central memory T cell (CD25+, CD45RA−, CD45RO+, CD127+), an effector T regulatory cell (Treg) (CD25+/−, CD45RA−, CD45RO+, CD127−, CTLA-4+), an effector memory Treg (CD25+, CD45RA−, CD45RO+, CD127+, CTLA-4+), a T memory stem cell (T_{SCM}) (CD45RA+, CCR7+, CD27+, CD95+CXCR3+), or a naïve T cell (CD25−, CD45R+−, CD45RO−, CD127+). In some embodiments, the T cell is a tumor infiltrating T cell (or TIL), or a T cell that is obtained from a tumor.

In some embodiments, the T cells having an increased level of an AMPKγ2 polypeptide have one or more characteristics as compared to a control T cell, those characteristics being selected from the group consisting of: 1) an increased oxidative metabolism, 2) less differentiated, 3) an increased extracellular acidification rate (ECAR), 4) an increased level of proliferation, 5) an increased in vivo survival time, and 6) an increased effector function. A cell's oxidative metabolism level and ECR can be determined by any method known to those of skill in the art. In some embodiments, the level of oxidative metabolism is equivalent to the oxidative consumption rate (OCR), which can be determined using, for example, a Seahorse Extracellular Flux Analyzer. ECAR can also be determined using a Seahorse Extracellular Flux Analyzer. In some embodiments, the T cell having an increased level of an AMPKγ2 polypeptide has an increased oxidative metabolism as compared to a control T cell. In some embodiments, the T cell having an increased level of an AMPKγ2 polypeptide is less differentiated than a control T cell. In some embodiments, the T cell having an increased level of an AMPKγ2 polypeptide has an increased ECAR as compared to a control T cell. In some embodiments, the T cell having an increased level of an AMPKγ2 polypeptide has an increased level of proliferation as compared to a control T cell. In some embodiments, the T cell having an increased level of an AMPKγ2 polypeptide has an increase in vivo survival time as compared to a control T cell. In some embodiments, the T cell having an increased level of an AMPKγ2 polypeptide has an increased effector function as compared to a control T cell.

In naive and memory T cells, mitochondria-dependent catabolic pathways, including glucose oxidation through the tricarboxylic acid (TCA) cycle and β-oxidation of fatty acids, provide most of the metabolic support for basic cellular functions. After T cell activation in vitro, β-oxidation rapidly decreases and other metabolic pathways, including glycolysis and glutaminolysis, increase. Multiple efforts have attempted to make cells more oxidative which aligns with their in vivo metabolism, a phenomenon well-known in the art. These alternative methods include limiting glycolysis, decreasing mitochondrial membrane potential, promoting mitochondrial fusion. In some embodiments, the T cell (e.g., a CD4+ T cell or a CD8⁺ T cell) disclosed herein has an increased oxidative metabolism as compared to the control. The increase in mitochondria-dependent oxidative metabolism can be determined by measuring the mitochondria membrane potential and mitochondrial mass of the T cell, or measuring oxygen consumption level (OCR) (e.g., using Seahorse machine) (van der Windt et al., 2016. Measuring bioenergetics in T cells using a seahorse extracellular flux analyzer. Curr. Protoc. Immunol. 113:3.16B.1-3.16B.14).

In some embodiments, the T cell (e.g., a CD4+ T cell or a CD8⁺ T cell) with an increased level of the AMPKγ2 polypeptide is less differentiated as compared to the control. It should be understood that, upon activation, a naïve T cell or a memory T cell expands and differentiates into different subsets. A CD4+ T cell can differentiate into, for example, a T helper type (Th) 1, Th2, Th9, Th17, Th22, or Tfh. A CD8⁺ T cell can differentiate into, for example, a cytotoxic T cell, short-lived effector cell, a central memory T cell, an effector memory T cell, or an exhausted T cell. Accordingly, in some embodiments, the T cell (e.g., a CD4+ T cell or a CD8⁺ T cell) with an increased level of the AMPKγ2 polypeptide using the method disclosed herein exhibits a less-differentiated or a naïve T cell-like phenotype, such as CD62L⁺CD45RA⁺, or CD62L⁺CD27⁺. The methods of determining a less-differentiated phenotype of a T cell are well-known in the art.

### Methods

As noted above, disclosed herein is a method of making a T cell comprising increasing a level of an AMPKγ2 polypeptide in the T cell as compared to a control. This method has been shown to be surprisingly effective at increasing T cell oxidative metabolism levels, increasing T cell in vivo persistence as compared to a control, increasing suppressive functionality in regulatory T cells, and/or increasing the amount or percentage of CD4+ T cells in a T cell population.

Accordingly, in some embodiments, disclosed herein is a method of making a T cell comprising increasing a level of an AMPKγ2 polypeptide in the T cell as compared to a control, wherein the T cell has one or more characteristics as compared to a control T cell, those characteristics being selected from the group consisting of: 1) an increased oxidative metabolism, 2) less differentiated, 3) an increased extracellular acidification rate (ECAR), 4) an increased level of proliferation, 5) an increased in vivo survival time, and 6) an increased effector function. Included herein are methods of making a T cell comprising increasing a level of an AMPKγ2 polypeptide in the T cell as compared to a control, wherein the T cell has an increased oxidative metabolism as compared to a control. Also included herein are methods of making a T cell comprising increasing a level of an AMPKγ2 polypeptide in the T cell as compared to a control, wherein the T cell is less differentiated than a control. Further included are methods of making a T cell comprising increasing a level of an AMPKγ2 polypeptide in the T cell as compared to a control, wherein the T cell has an increased extracellular acidification rate (ECAR). Still further included are methods of making a T cell comprising increasing a level of an AMPKγ2 polypeptide in the T cell as compared to a control, wherein the T cell has an increased level of proliferation. Also included are methods of making a T cell comprising increasing a level of an AMPKγ2 polypeptide in the T cell as compared to a control, wherein the T cell has an increased in vivo survival time. Also included are methods of making a T cell comprising increasing a level of an AMPKγ2 polypeptide in the T cell as compared to a control, wherein the T cell has an increased effector function.

In some embodiments, the AMPKγ2 polypeptide comprises SEQ ID NO:1. In some embodiments, the AMPKγ2 polypeptide comprises SEQ ID NO: 3. In some embodiments, the AMPKγ2 polypeptide comprises a polypeptide sequence having at or greater than about 80%, about 85%, about 90%, about 95%, or about 98% homology or identity with SEQ ID NO:1 or SEQ ID NO: 3, or a polypeptide comprising a portion of SEQ ID NO:1 or SEQ ID NO: 3.

In some aspects, the method of making a T cell comprises increasing a level of an AMPKγ2 polypeptide in the T cell as compared to the control, wherein the T cell is a Treg and has an increased suppressor function as compared to a control. It should be understood that the term “an increased suppressor function as compared to a control” refers to the T cell having an increased ability to suppress proliferation, cytokine production and/or up-regulation of activation markers in other cells, including other T cells. In some embodiments, the Treg expresses increased levels of CD25 and FoxP3 and/or produces more IL-10 in comparison to a control wherein the control has not been transduced with an AMPKγ2 polynucleotide. In some embodiments, the AMPKγ2 polypeptide comprises SEQ ID NO:1. In some embodiments, the AMPKγ2 polypeptide comprises SEQ ID NO: 3. In some embodiments, the AMPKγ2 polypeptide comprises a polypeptide sequence having at or greater than about 80%, about 85%, about 90%, about 95%, or about 98% homology with SEQ ID NO:1 or SEQ ID NO: 3, or a polypeptide comprising a portion of SEQ ID NO:1 or SEQ ID NO: 3.

In some embodiments, the method further comprises culturing the T cell in the presence of one or more cytokines (e.g., IL-2). In some embodiments, the T cell is cultured for about 2 to 12 days. Culturing T cells can lead to T cell expansion.

Included herein is a method of making a regulatory T cell comprising increasing a level of a AMPKγ2 polypeptide in the regulatory T cell as compared to the control, wherein the AMPKγ2 polypeptide comprising a sequence at least 95% identical to SEQ ID NO:1, SEQ ID NO: 3 or a fragment thereof, and wherein the regulatory T cell has an increased suppressor function as compared to a control. In some embodiments, the AMPKγ2 polypeptide comprises a sequence at least 97% identical to SEQ ID NO:1 or SEQ ID NO: 3. In some embodiments, the AMPKγ2 polypeptide comprises a sequence at least 99% identical to SEQ ID NO:1 or SEQ ID NO: 3.

In some embodiments, the level of the AMPKγ2 polypeptide is increased via transducing a vector into the T cell, wherein the vector comprises a polynucleotide sequence disclosed herein for encoding the AMPKγ2 polypeptide. In some embodiments, the AMPKγ2 polynucleotide sequence encodes an AMPKγ2 polypeptide that comprises SEQ ID NO:1. In some embodiments, the AMPKγ2 polynucleotide sequence encodes an AMPKγ2 polypeptide that comprises SEQ ID NO: 3. In some embodiments, the AMPKγ2 polynucleotide sequence encodes an AMPKγ2 polypeptide that comprises a polypeptide sequence having at or greater than about 80%, about 85%, about 90%, about 95%, or about 98% homology with SEQ ID NO:1 or SEQ ID NO: 3, or a polypeptide comprising a portion of SEQ ID NO:1 or SEQ ID NO: 3. In some embodiments, the AMPKγ2 polynucleotide sequence is at least about 80%, about 85%, about 90%, about 95%, about 97%, about 98%, about 99%, about 99.5% identical to SEQ ID NO: 2, SEQ ID NO: 4, or a fragment thereof. The control can be a T cell that has not been transduced with an AMPKγ2 polynucleotide.

In some aspects, disclosed herein is a method of making a population of T cells comprising increasing a level of a AMPKγ2 polypeptide in the T cells as compared to the control, and wherein the population has an increased amount of CD4+ T cells and has one or more characteristics as compared to a control T cell population, those characteristics being selected from the group consisting of: 1) an increased oxidative metabolism, 2) less differentiated, 3) an increased extracellular acidification rate (ECAR), 4) an increased level of proliferation, 5) an increased in vivo survival time, and 6) an increased effector function. In some embodiments, the AMPKγ2 polypeptide comprises SEQ ID NO: 3. In some embodiments, the AMPKγ2 polypeptide comprises a polypeptide sequence having at or greater than about 80%, about 85%, about 90%, about 95%, or about 98% homology with SEQ ID NO:1 or SEQ ID NO: 3, or a polypeptide comprising a portion of SEQ ID NO:1 or SEQ ID NO: 3. The control can be a population of T cells that have not been transduced with an AMPKγ2 polynucleotide.

In some embodiments, the method further comprises culturing the T cells in the presence of IL-2. In one example, the T cell is cultured for about 2 to 12 days (for example, for about 2 days, 3 days, 4 days, 5 days, 6 days, 7 days, 8 days, 9 days, 10 days, 11 days, or 12 days). It should be understood that the increased amount of CD4⁺ T cells refers to an increase in CD4⁺ T cell numbers prior to or after culturing the T cells, or after administering the T cells to a subject. In some embodiments, the increased amount of CD4⁺ T cells refers to an increase in the CD4⁺ T cell:CD8⁺ T cell ratio prior to or after culturing the T cells, or after administering the T cells to a subject, as compared to the control T cell population not having been transduced with an AMPKγ2 polynucleotide.

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). See also International Patent Publication No. WO2012079000A1, incorporated by reference herein in its entirety. Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

In some embodiments, the vector used in the methods is a viral vector. “Viral vector” as disclosed herein means, in respect to a vehicle, any virus, virus-like particle, virion, viral particle, or pseudotyped virus that comprises a nucleic acid sequence that directs packaging of a nucleic acid sequence in the virus, virus-like particle, virion, viral particle, or pseudotyped virus. In some embodiments, the virus, virus-like particle, virion, viral particle, or pseudotyped virus is capable of transferring a vector (such as a nucleic acid vector) into and/or between host cells. In some embodiments, the virus, virus-like particle, virion, viral particle, or pseudotyped virus is capable of transferring a vector (such as a nucleic acid vector) into and/or between target cells, such as a hepatocyte in the liver of a subject. Importantly, in some embodiments, the virus, virus-like particle, virion, viral particle, or pseudotyped virus is capable of transporting into a nucleus of a target cell (e.g., a hepatocyte). The term “viral vector” is also meant to refer to those forms described more fully in U.S. Patent Application Publication U.S. 2018/0057839, which is incorporated herein by reference for all purposes. Suitable viral vectors include, e.g., adenoviruses, adeno-associated virus (AAV), vaccinia viruses, herpesviruses, baculoviruses and retroviruses, parvoviruses, and lentiviruses. In some embodiments, the viral vector is a lentiviral vector.

Since the compositions and methods described herein can be used to increase the in vivo persistence of a T cell, contemplated herein are methods for treating any disease or condition wherein T cells are administered to a subject for such treatment. Also included herein are method of increasing an immune response in a subject comprising administering to the subject a therapeutically effective amount of a T cell having an increased level of an AMPKγ2 polypeptide as compared to a control. In some embodiments, the methods comprise a form of adoptive T cell therapy wherein T cells are removed from a subject, transduced with a vector comprising an AMPKγ2 encoding polynucleotide, and the transduced T cells are then returned to the subject.

The T cell of the adoptive T cell therapy methods can be of any type, including, but not limited to, a CD4+ T cell or a CD8⁺ T cell. The CD4+ T cell can be a T helper cell (Th), a regulatory T cell (Treg) or a follicular T helper cell (Tfh). The CD4+T helper T cell can be a Th1, a Th2, a Th9, a Th17, or a Th22. For example, Th1 releases IFN-7 and TNF; Th2 releases TL-4 (an important survival factor for B-type lymphocytes), IL-5 and IL-13; Th9 produces IL-9; Treg secretes IL-10 (a cytokine with an immunosuppressive function, maintaining expression of FOXP3 transcription factor needed for suppressive function of Treg on other cells) and TGF-β; and Th17 produces IL-17 (a cytokine playing an important role in host defense against bacteria, and fungi). In some aspects, the T cell is an effector T cell (CD25+, CD45RA+/−, CD45RO+/−, CD127−), an effector memory T cell (CD25−, CD45RA−, CD45RO+, CD127+), a T_{EMRA} (CD25-, CD45RA+, CD45RO+, CD127+), a central memory T cell (CD25+, CD45RA−, CD45RO+, CD127+), an effector T regulatory cell (Treg) (CD25+/−, CD45RA−, CD45RO+, CD127−, CTLA-4+), an effector memory Treg (CD25+, CD45RA−, CD45RO+, CD127+, CTLA-4+), a T memory stem cell (T_{SCM}) (CD45RA+, CCR7+, CD27+, CD95+CXCR3+), or a naïve T cell (CD25−, CD45R+−, CD45RO−, CD127+). In some embodiments, the T cell is a tumor infiltrating T cell, or a T cell that is obtained from a tumor.

In some embodiments of the adoptive T cell therapy methods, the AMPKγ2 polypeptide comprises a sequence at least 90% identical to SEQ ID NO:1, SEQ ID NO: 3, or a fragment thereof. In some embodiments, the transduced T cells are cultured in the presence of one or more cytokines (e.g., IL-2) before being reintroduced to the subject. In some embodiments, the T cell is cultured for about 2 to 12 days.

Examples of increased immune responses can be increased killing effects on a target, such as a tumor, a pathogen or a vaccine, or increased suppressive effects on inflammation or autoimmunity. Accordingly, in some embodiments, the method of increasing an immune response can be used to treat cancers or infections. In these embodiments, treatment can be a reduction in the size of a tumor, of the number of tumors, and/or in the metastasis of a tumor in the subject. In some embodiments, the method of increasing an immune response can be used to treat diseases relating to increased inflammation, such as an inflammatory disease or autoimmune diseases.

In one aspect, the methods described herein are used to treat cancer, for example, melanoma, lung cancer (including lung adenocarcinoma, basal cell carcinoma, squamous cell carcinoma, large cell carcinoma, bronchioloalveolar carcinoma, bronchogenic carcinoma, non-small-cell carcinoma, small cell carcinoma, mesothelioma); breast cancer (including ductal carcinoma, lobular carcinoma, inflammatory breast cancer, clear cell carcinoma, mucinous carcinoma, serosal cavities breast carcinoma); colorectal cancer (colon cancer, rectal cancer, colorectal adenocarcinoma); anal cancer; pancreatic cancer (including pancreatic adenocarcinoma, islet cell carcinoma, neuroendocrine tumors); prostate cancer; prostate adenocarcinoma; ovarian carcinoma (ovarian epithelial carcinoma or surface epithelial-stromal tumor including serous tumor, endometrioid tumor and mucinous cystadenocarcinoma, sex-cord-stromal tumor); liver and bile duct carcinoma (including hepatocellular carcinoma, cholangiocarcinoma, hemangioma); esophageal carcinoma (including esophageal adenocarcinoma and squamous cell carcinoma); oral and oropharyngeal squamous cell carcinoma; salivary gland adenoid cystic carcinoma; bladder cancer; bladder carcinoma; carcinoma of the uterus (including endometrial adenocarcinoma, ocular, uterine papillary serous carcinoma, uterine clear-cell carcinoma, uterine sarcomas, leiomyosarcomas, mixed mullerian tumors); glioma, glioblastoma, medulloblastoma, and other tumors of the brain; kidney cancers (including renal cell carcinoma, clear cell carcinoma, Wilm's tumor); cancer of the head and neck (including squamous cell carcinomas); cancer of the stomach (gastric cancers, stomach adenocarcinoma, gastrointestinal stromal tumor); testicular cancer; germ cell tumor; neuroendocrine tumor; cervical cancer; carcinoids of the gastrointestinal tract, breast, and other organs; signet ring cell carcinoma; mesenchymal tumors including sarcomas, fibrosarcomas, haemangioma, angiomatosis, haemangiopericytoma, pseudoangiomatous stromal hyperplasia, myofibroblastoma, fibromatosis, inflammatory myofibroblastic tumor, lipoma, angiolipoma, granular cell tumor, neurofibroma, schwannoma, angiosarcoma, liposarcoma, rhabdomyosarcoma, osteosarcoma, leiomyoma, leiomysarcoma, skin, including melanoma, cervical, retinoblastoma, head and neck cancer, pancreatic, brain, thyroid, testicular, renal, bladder, soft tissue, adenal gland, urethra, cancers of the penis, myxosarcoma, chondrosarcoma, osteosarcoma, chordoma, malignant fibrous histiocytoma, lymphangiosarcoma, mesothelioma, squamous cell carcinoma; epidermoid carcinoma, malignant skin adnexal tumors, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma anaplastic; glioblastoma multiforme, neuroblastoma, medulloblastoma, malignant meningioma, malignant schwannoma, neurofibrosarcoma, parathyroid carcinoma, medullary carcinoma of thyroid, bronchial carcinoid, pheochromocytoma, Islet cell carcinoma, malignant carcinoid, malignant paraganglioma, melanoma, Merkel cell neoplasm, cystosarcoma phylloide, salivary cancers, thymic carcinomas, and cancers of the vagina among others. In some embodiments the cancer is a leukemia. In some aspects, the cancer is acute myelogenous leukemia or acute lymphoblastic leukemia.

In these methods, the dosage forms of the compositions disclosed herein can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavenous, intrathecal, intravitreal, intracerebral, gingival, subgingival, intracerebroventricular, and intradermal. Such formulations may be prepared by any method known in the art.

The disclosed methods can be performed any time prior to and/or after the onset of a disease (e.g., a cancer or an infection) or administration of a vaccine. In some aspects, the disclosed methods can be employed 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 years; 12, 11, 10, 9, 8, 7, 6, 5, 4, 3, 2, or 1 months; 30, 29, 28, 27, 26, 25, 24, 23, 22, 21, 20, 19, 18, 17, 16, 15, 14, 13, 12, 11, 10, 9, 8, 7, 6, 5, 4, or 3 days; 60, 48, 36, 30, 24, 18, 15, 12, 10, 9, 8, 7, 6, 5, 4, 3, or 2 hours prior to the onset of a disease (e.g., a cancer or an infection) or administration of a vaccine; or 1, 2, 3, 4, 5, 6, 7, 8, 9, 10, 15, 20, 25, 30, 35, 40, 45, 50, 55, 60, 75, 90, 105, 120 minutes; 3, 4, 5, 6, 7, 8, 9, 10, 11, 12, 15, 18, 24, 30, 36, 48, 60 hours; 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days; 4, 5, 6, 7, 8, 9, 10, 11, 12 or more months; 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 years after the onset of a disease (e.g., a cancer or an infection) or administration of a vaccine.

Dosing frequency for the T cell compositions disclosed herein, includes, but is not limited to, at least once every 12 months, once every 11 months, once every 10 months, once every 9 months, once every 8 months, once every 7 months, once every 6 months, once every 5 months, once every 4 months, once every 3 months, once every two months, once every month; or at least once every three weeks, once every two weeks, once a week, twice a week, three times a week, four times a week, five times a week, six times a week, or daily. In some embodiment, the interval between each administration is less than about 4 months, less than about 3 months, less than about 2 months, less than about a month, less than about 3 weeks, less than about 2 weeks, or less than less than about a week, such as less than about any of 6, 5, 4, 3, 2, or 1 day. In some embodiment, the dosing frequency for the T cells disclosed herein includes, but is not limited to, at least once a day, twice a day, or three times a day. In some embodiment, the interval between each administration is less than about 48 hours, 36 hours, 24 hours, 22 hours, 20 hours, 18 hours, 16 hours, 14 hours, 12 hours, 10 hours, 9 hours, 8 hours, or 7 hours. In some embodiment, the interval between each administration is less than about 24 hours, 22 hours, 20 hours, 18 hours, 16 hours, 14 hours, 12 hours, 10 hours, 9 hours, 8 hours, 7 hours, or 6 hours. In some embodiment, the interval between each administration is constant. For example, the administration can be carried out daily, every two days, every three days, every four days, every five days, or weekly. Administration can also be continuous and adjusted to maintaining a level of the compound within any desired and specified range.

All patents, patent applications, and publications referenced herein are incorporated by reference in their entirety for all purposes.

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1

Acute myelogenous and acute lymphoblastic leukemia (AML/ALL) remain prevalent cancers in both children and adults, including in service men and women exposed to ionizing radiation. Developing myeloma is another risk of radiation exposure. Furthermore, many of these cancers pose difficult treatment challenges. Approaches which use primary immune cells to target antigens expressed predominantly on cancer cells have the potential to improve leukemia and myeloma control and provide long-term cures. However, full translation of this game changing technology is hindered by an inability of in vitro activated T cells to survive well in vivo. Similarly, transfer of autologous T cells bearing chimeric antigen receptors (CARs) has revolutionized the treatment of high-risk and refractory acute lymphoblastic leukemia. However, a significant subset of patients receiving CAR T cells continue to suffer relapse or incomplete responses due to a lack of CAR T cell persistence. Increasing the in vivo persistence of tumor-reactive lymphocytes can improve tumor clearance. The increased immune cell persistence can be achieved through directed expression of metabolic proteins specifically in the T cells of interest. Thus, the present study shows the methods for improving anti-cancer immune responses by enhancing the in vivo persistence of tumor-reactive lymphocytes.

It has been found that T cells expanding rapidly in vivo up-regulate both the metabolic energy sensor, AMP-activated protein kinase (AMPK). Furthermore, absence of either gene in donor T cells decreases their proliferation and survival in allogeneic recipients, indicating that both proteins are necessary for optimal T cell expansion in vivo. These data indicate that constitutive activation of AMPK, can enhance the metabolic fitness of cultured T cells, leading to prolonged anti-leukemia responses in vivo.

The present study shows metabolic changes in human T cells following constitutive activation of AMPK. The lab has recently generated lentiviral constructs bearing either wildtype or mutant AMPKγ2 sequences, which drive AMPK activation. The mutant AMPKγ2 sequences contained mutations at position 488 (N488I) and position 531 (R531G). Lentiviral genes are transduced into human T cells and T cell metabolism assessed following in vitro and in vivo stimulation. The impact of constitutive AMPK activation on T cell persistence and subsequent anti-tumor responses in vivo is determined. CAR T cells, targeting human CD19, are transduced with a second lentivirus bearing wildtype or mutant AMPKγ2. CAR T cells, with or without metabolic manipulation, are then injected with CD19+ leukemia cells into immunodeficient mice, where both T cell persistence and anti-tumor responses are measured. It was determined that the wildtype AMPKγ2 sequences provided better results than the mutant AMPKγ2 sequences. In a second model, cytotoxic lymphocytes (CTLs) are generated against the antigen PR-1. PR1-reactive CTLs are then transduced with metabolic lentiviral constructs and injected into immunodeficient mice, followed by assessment of CTL persistence and the ability to clear a PR1-presenting tumor cell line. In a final series of experiments, primary human T cells are expanded in vitro against primary human AML blasts, transduced with AMPK lentiviral constructs, and then tested for the ability to eliminate primary AML cells in vivo in an immunodeficient mouse model.

Despite marked improvements in cancer care, some blood cancers, remain formidable treatment challenges. Immune cell targeting of tumor antigens offer hope for these difficult cases, but the full potential of adoptive immunotherapy remains unrealized. The present study comes from taking a recognized clinical problem (a lack of immune cell persistence) and addressing it by metabolically reprogramming an established treatment. The result can positively impact the wider field of immunotherapy by determining whether metabolic changes, such as constitutive activation of AMPK, can improve T cell responses by prolonging their survival and thereby lessening overall disease burden. These studies are also important for cancer research in general, as the ability to modulate immune responses can be a cardinal feature in multiple treatment regimens. Clinically, improved treatments for difficult-to-treat cancers is relevant to the American public, not only because of the sheer number of leukemia and myeloma cases diagnosed each year, but also because of the personal impact that each diagnosis brings.

It has become clear that effector T cells which are capable of performing oxidative metabolism are more “fit” for the metabolic conditions they subsequently encounter in vivo such that finding ways to promote oxidative metabolism in T cells increases their in vivo persistence. Furthermore, there is an inverse correlation between a how terminally differentiated a cell becomes and the likelihood that this cell adopts oxidative metabolism (i.e. differentiated effector cells are unlikely to be oxidative). Thus, a major goal in the field of cellular therapies is to define ways to make effector cells both less-differentiated and simultaneously more oxidative during their ex vivo expansion or upon their transfer into recipients.

Multiple efforts have attempted to make cells more oxidative. These include limiting glycolysis, decreasing mitochondrial membrane potential, inhibiting Akt activity, promoting mitochondrial fusion, or inducing alternative cytokine signaling (e.g. interleukin-21). However, consistently making cells less differentiated and more oxidative has proven difficult, complicated by the fact that many metabolic perturbations, such as treatment of cells with the glucose analog 2-deoxyglucose, often come at the expense of reduced cell growth and decreased in vitro expansion. In addition, the use of metabolic inhibitors raises the potential concern for off target-effects. For example, treatment of T cells with the diabetes drug metformin activates the cellular energy sensor AMP-activated protein kinase (AMPK). This result is expected to increase oxidative metabolism as AMPK works upstream in many of these pathways. However, metformin increases AMPK activity indirectly by blocking the electron transport chain and making cells “feel starved”. Thus, the use of metformin, instead of increasing oxidative metabolism by driving AMPK activation, in reality decreases a cell's ability to use oxygen in the creation of energy. As alluded to above, increasing AMPK can increase the oxidative metabolism of a cell. In effector T cells, such as those trained to fight cancer, this increase in oxidative metabolism can lead to their increased in vivo persistence.

The exact same increase in oxidative metabolism driven by AMPK in Tregs can lead to an increase in both their stability and suppressive function, functioning in both Tregs and T effector cells as a kind of oxidative insurance policy. The present study shows that modulating AMPK activity can improve the efficacy of multiple therapeutic interventions dependent upon T cells.

To overcome the challenges of decreased in vivo persistence of effector T cells, or the decreased suppressive function or stability of Treg, the regulatory component of the AMPK heterotrimeric complex is overexpressed via lentiviral transduction of primary human T cells. Importantly, similar lentiviral technology is already used in the clinic to generate cancer-targeting CAR T cells. AMPK is a heterotrimeric protein complex consisting of an a domain, containing the kinase activity, and a regulatory γ subunit, which controls the ability of the α domain to become phosphorylated and thus activated. Indeed, it is AMPKγ-regulated phosphorylation of AMPKα which largely controls the kinase activity of the complex. By over-expressing a truncated version of human AMPKγ2, AMPK signaling is significantly upregulated in both T cell lines and primary human T cells. Furthermore, cells transduced with AMPKγ2 exhibited both a less differentiated, more central memory-like phenotype and an increase in mitochondrial and oxidative metabolism. Furthermore, constitutive activation of AMPK can improve the in vivo persistence of ex vivo and in vitro manipulated effector T cells as well as increase the stability and suppressive functionality of Treg cells.

### Example 2

Multiple applications of improving T cell oxidative metabolism: Contexts where this invention can be beneficial include increased persistence of cancer targeting T cells (CAR T cells, tumor-infiltrating lymphocytes, T cells bearing a cloned T cell receptor), improved T cell responses during vaccination, and cellular therapies involving Tregs including treatment or prevention of autoimmune diseases like Type 1 diabetes and alloreactive responses like those seen during GVHD or solid organ transplant rejection.

### Example 3

Durability of manipulation: As mentioned above, current metabolic manipulations rely on brief pharmacologic treatment of T cells during ex vivo expansion, hoping that this exposure engenders lasting change. However, once treated cells are administered, the cells are no longer exposed to pharmacologic pressure and may easily revert their phenotype back to something less oxidative and potentially less metabolically fit. The approach shown herein was designed to utilize genetic integration of the AMPK γ² sequence into the T cells, meaning that the transduced cells, as well as their progeny, are endowed with these metabolic enhancements for the life of the cell, whether in vitro or in vivo.

### Example 4

Improved specificity: As noted previously, pharmacologic interventions invariably carry the potential for off-target effects or on-target effects in off-target pathways. In addition, like metformin they may impact metabolism indirectly. The approach shown herein to increase AMPK signaling avoids these challenges by introducing a well-known and well-characterized member of the AMPK protein complex. Because of the specific nature of the introduction, where and how this change works is known in the transduced cells. This improved specificity lowers the chances of unknown or unwanted side effects associated with pharmacological treatments.

### Example 5

Increased ex vivo expansion of manipulated T cells: An additional caveat to metabolic manipulation is that many interventions limit cell growth and expansion. For example, both glucose restriction and treatment of cells with the glycolysis inhibitor 2-DG increase oxidative metabolism. However, these interventions severely limit the growth of T cells during their in vitro expansion. Thus, while the appropriate metabolism is achieved, translating this approach clinically remains challenging as too few cells are recovered for reliable in vivo transfer. In contrast, the data of the present study show that constitutive expression of AMPKγ2 results in both less-differentiated cells with heightened oxidative metabolism and increased numbers of AMPKγ2-expressing cells following in vitro culturing.

### Example 6

Improved CD4 to CD8 T cell ratios. There are two broad subtypes of human T cells important for T cell therapies, CD4+ helper T cells and CD8+ cytotoxic T cells. Studies have shown that the best anti-cancer results occur with a near equal mix of CD4+ and CD8+ cells, in part because CD4+ T cells help tumor-targeting CD8+ T cells function more effectively. However, standard in vitro T cell expansion methods preferentially increase CD8+ cell numbers, disrupting the optimal balance between CD4+ and CD8+ T cells. The present study shows that constitutive expression of AMPKγ2 increases CD4 T cell percentages following in vitro expansion, in addition to increasing overall T cell numbers. This feature helps further improve the in vivo functionality of any T cell therapy that relies on both CD4+ and CD8+ T cell responses.

### Example 7

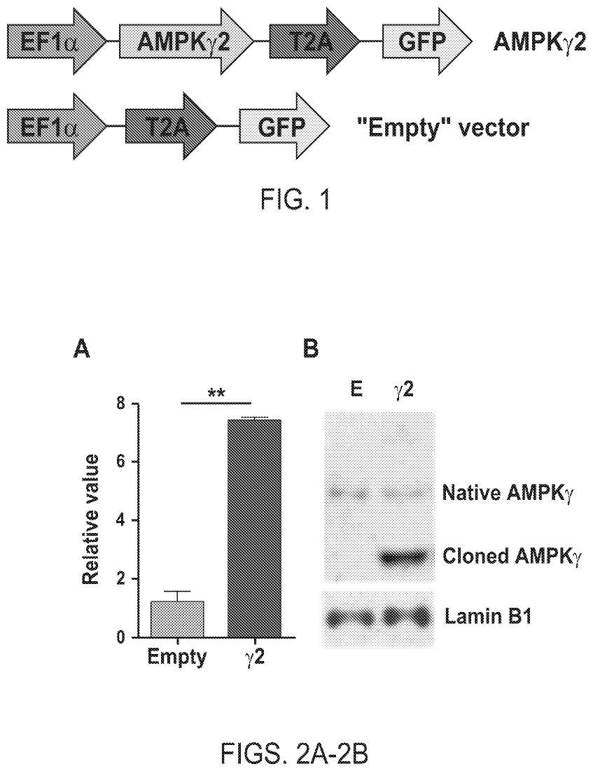

The present study uses naturally occurring AMPKγ2 sequences (cloned from humans) that constitutively increase AMPK activity (FIG. 1). First, expression of GFP-only (Empty) and AMPKγ2 constructs was determined in the Jurkat T cell line using a) flow cytometry to indicate GFP expression, b) RT-PCR to show transcript expression at the RNA level, and c) Western blot data to show production of the truncated AMPKγ2 protein (SEQ ID NO: 1). The data in FIGS. 2A-2C show the expression AMPKγ2 in Jurkat T cells. The lentiviral vector used in the studies was purchased from Addgene, Watertown, Massachusetts, United States; catalog number 31847; vector titled pSicoR-Ef1a-mCh.

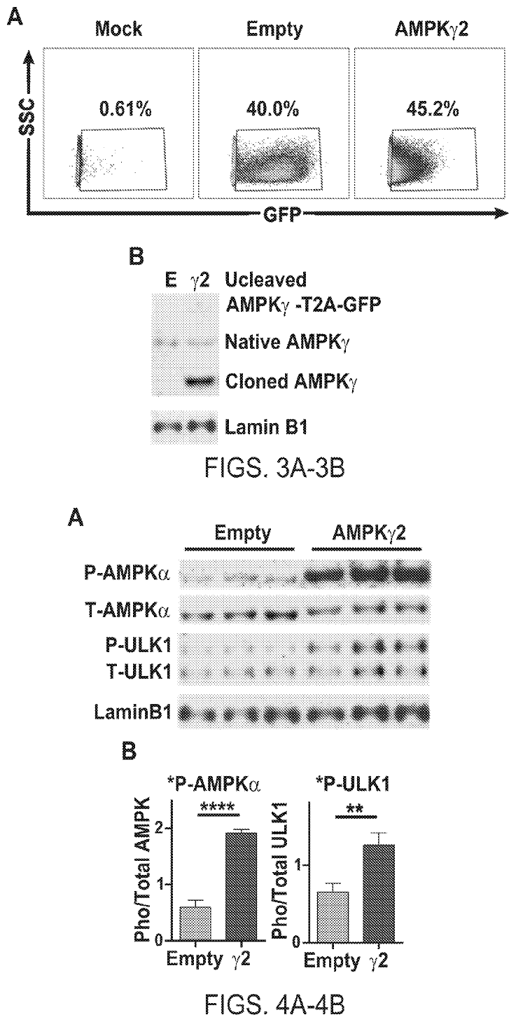

The next experiment determined GFP and AMPKγ2 protein expression in primary human T cells. Flow cytometry analysis (FIG. 3A) and Western blot data (FIG. 3B) show the truncated AMPKγ2 protein (SEQ ID NO: 1).

The next experiment investigated activation of AMPK by measuring phosphorylation of AMPKα and ULK-1 in AMPKγ2-transduced primary T cells. Increased phosphorylation of AMPKα and phosphorylation of ULK-1, a downstream target, was shown by immunoblot (FIG. 4A). FIG. 4B shows quantification of the Phospho/total AMPKα or phospho/total ULK-1 ratios as determined by densitometry analysis.

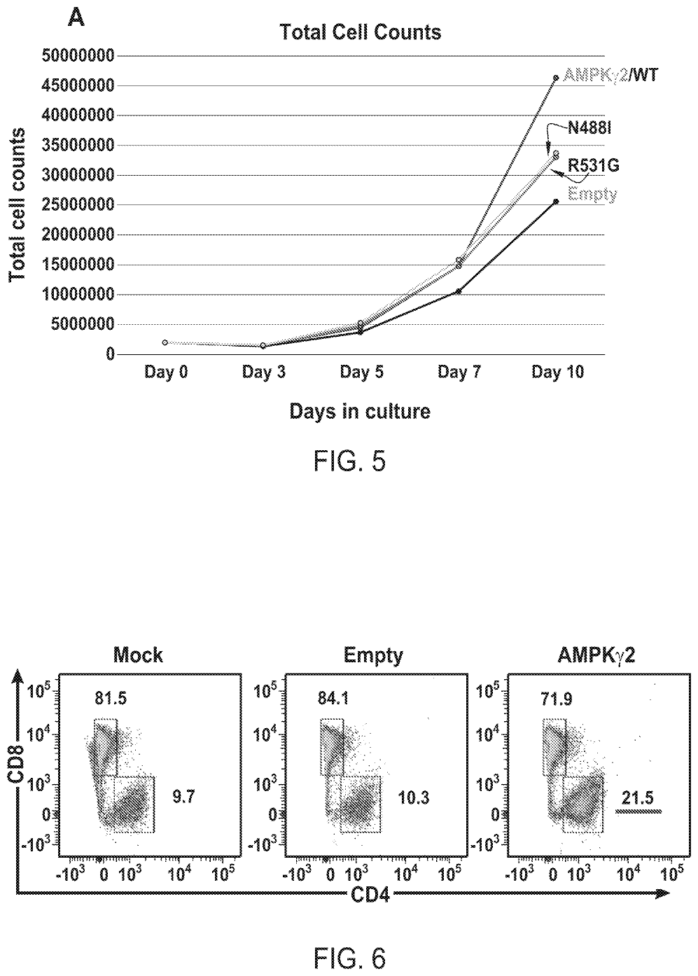

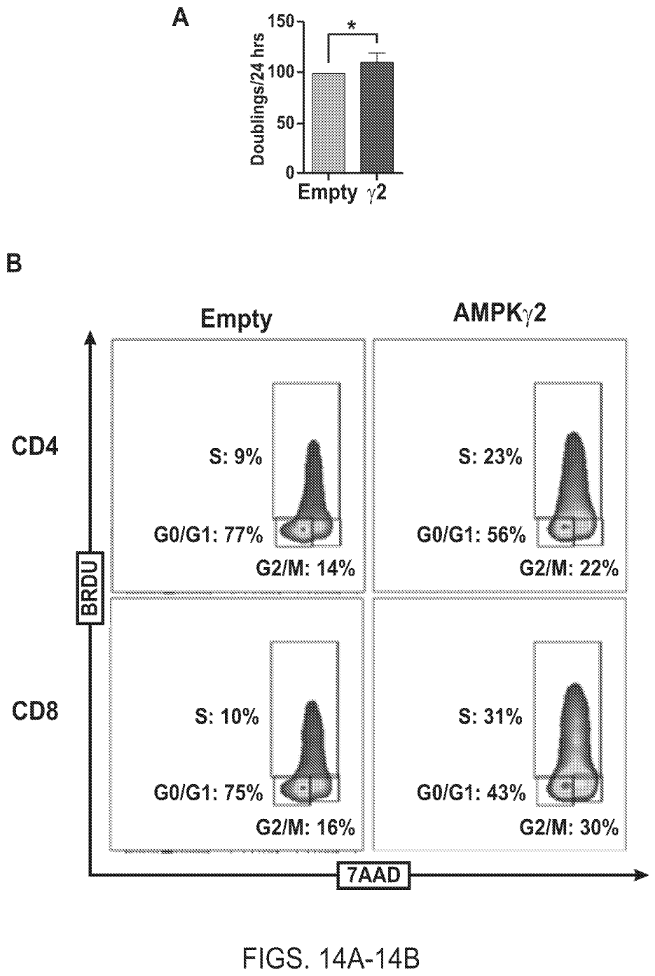

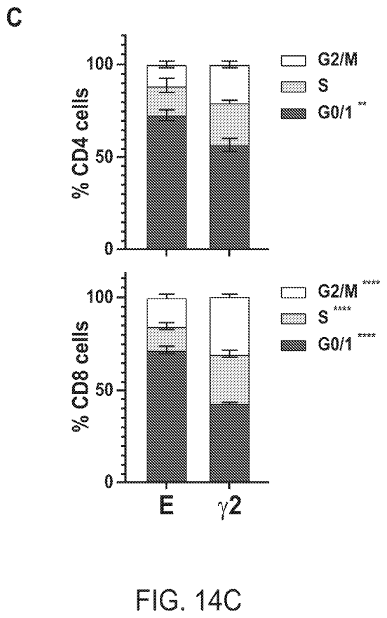

Concerns remained regarding whether AMPKγ2-transduction might decrease the growth of primary T cells. To investigate this, the total number of cells growing in cultures were counted following transduction of either empty constructs or those containing AMPKγ2. T cells transduced with wild type and truncated AMPKγ2 (SEQ ID NO: 1) exhibited increased growth in comparison with empty construct-transduced T cells (FIG. 5). This increased growth was also shown in FIGS. 14A-C.

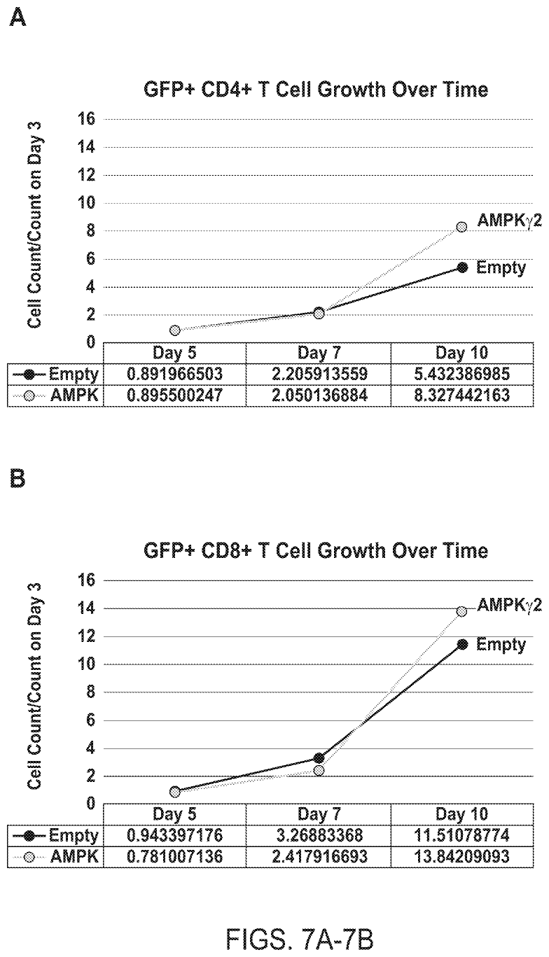

The ratio of CD4+ T cells (T helper cells) to CD8+ T cells (cytotoxic T cells) following AMPKγ2-transduction of primary human T cells was next quantitated. An increase in CD4+ T cell percentages was observed following AMPKγ2-transduction (FIG. 6). This result has been extremely reproducible, occurring in every transduction that was performed (multiple donors, multiple batches of virus, etc). To help explain this increase in CD4+ percentages, the growth of CD4+ and CD8+ T cells was then measured over time. Consistent with the increased percentages of CD4+ T cells, an increase in CD4+ T cell counts was also observed (FIG. 7).

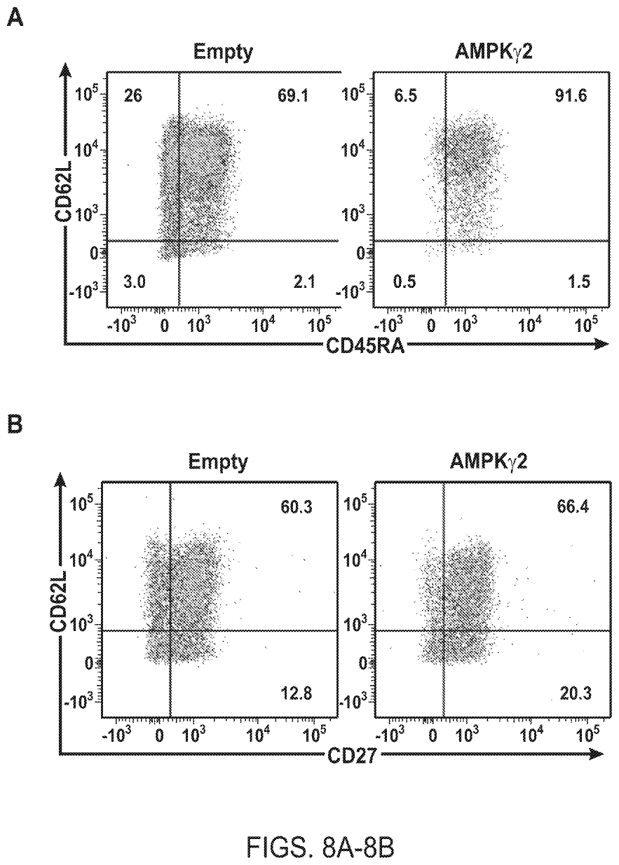

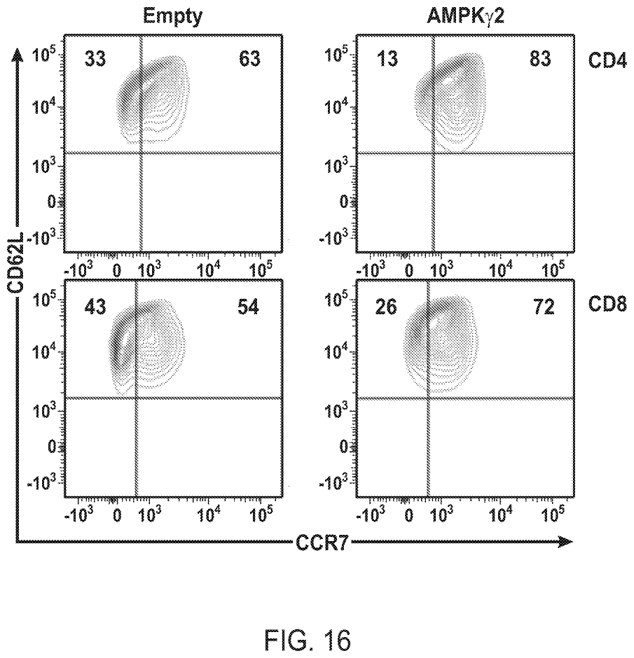

Whether transduced primary T cells remained less differentiated was next investigated. CD62L versus CD45RA expression at day 7 (FIG. 8A) and CD62L versus CD27 expression (FIG. 8B) at day 14 were measured. CD62L versus CCR7 was also measured in day 7 empty versus AMPKγ2-transduced T cells (FIG. 16). In all cases, the least differentiated cells reside in the upper right quadrant of the flow plots, indicating AMPKγ2-transduced T cells are less differentiated as measured by multiple parameters.

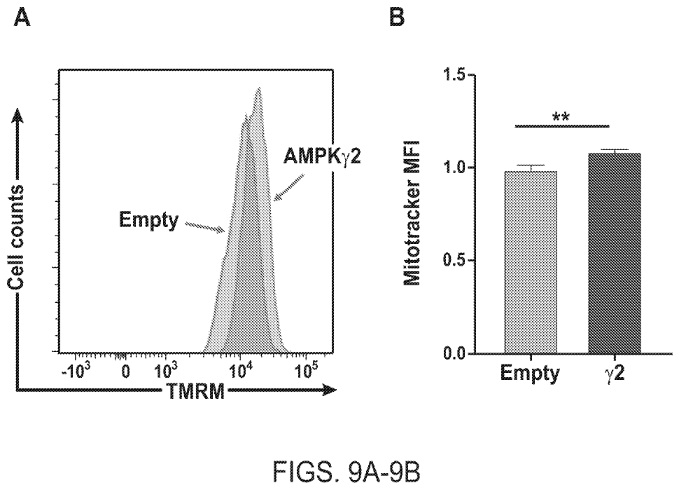

The next experiment assessed whether transduced primary T cells had increases in mitochondrial metabolism, which would support an increase in oxidative metabolism. A flow cytometry plot shows the difference in TMRM staining—a dye used to measure mitochondrial membrane potential/activity (FIG. 9A). Mitochondrial mass was also measured in empty versus AMPKγ2-transduced T cells in multiple replicates (FIG. 9B). Both sets of data show increased mitochondrial metabolism in AMPKγ2-transduced T cells.

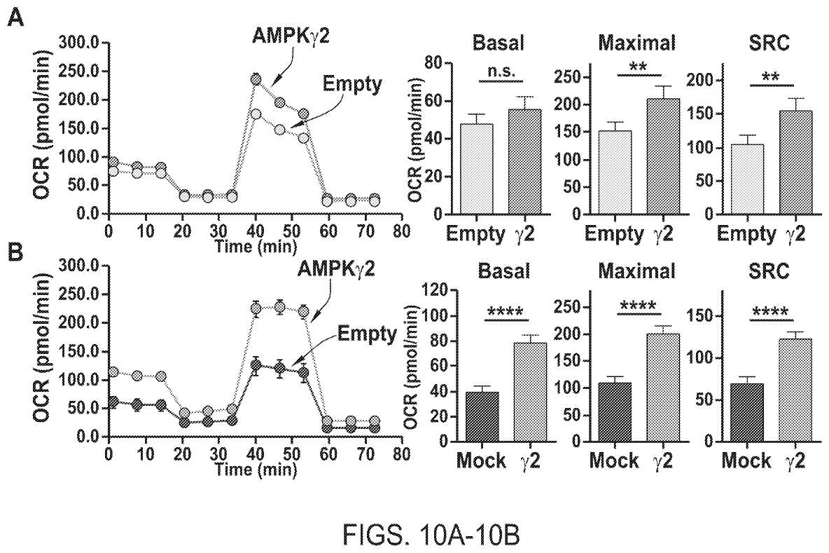

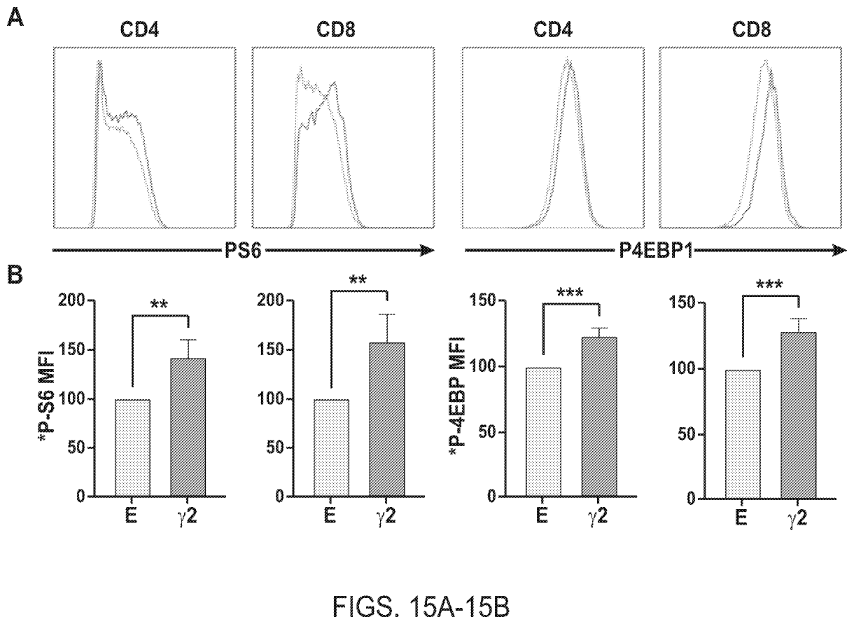

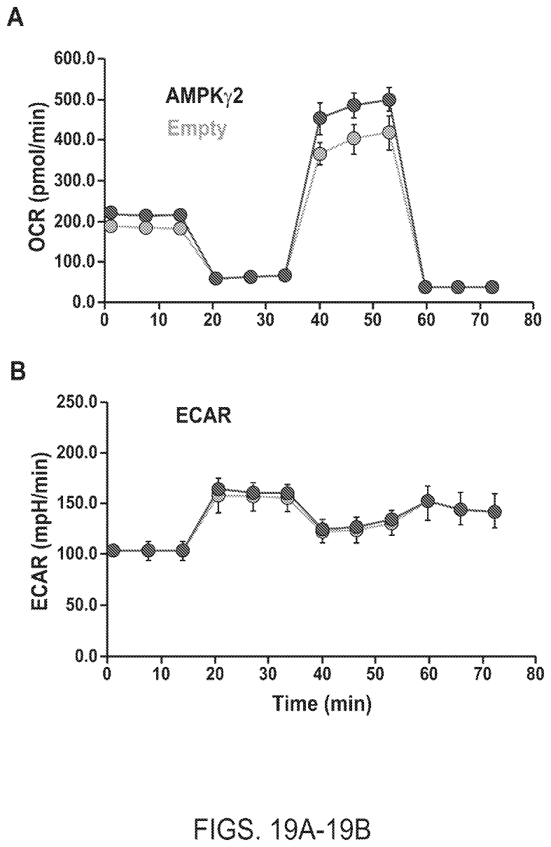

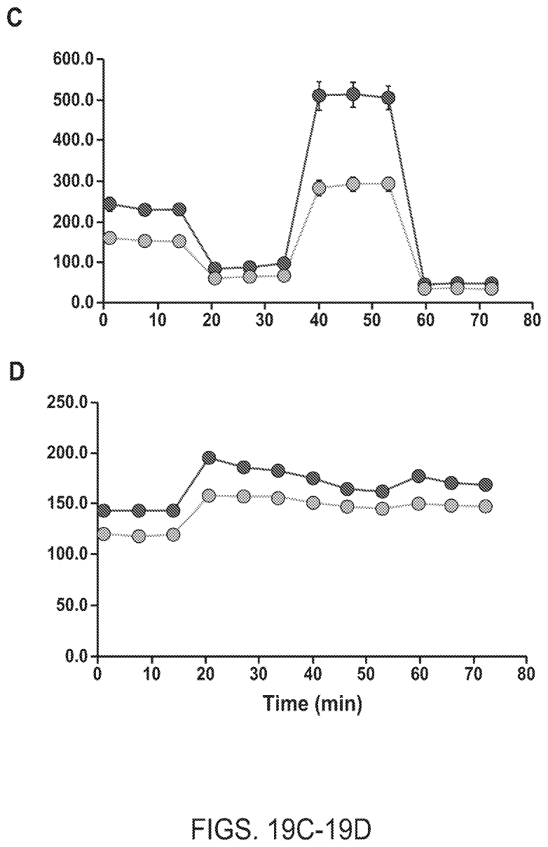

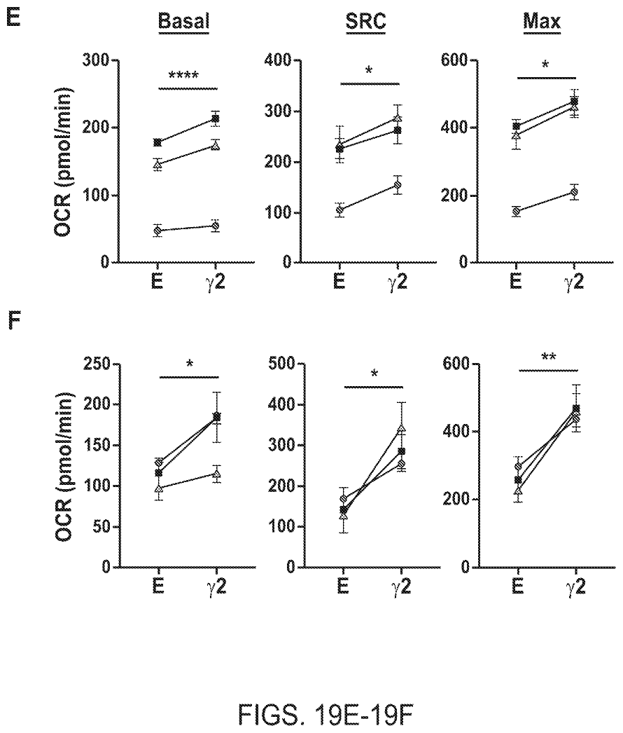

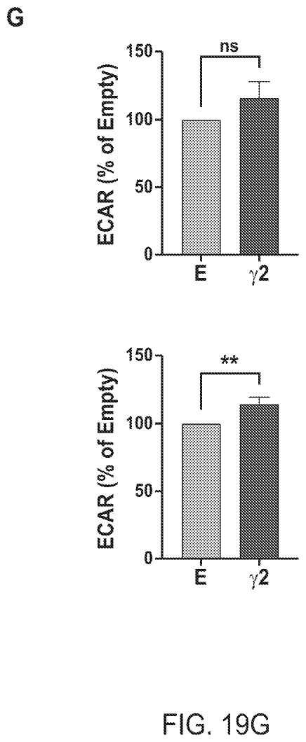

Oxygen consumption in transduced primary T cells was next investigated using a Seahorse metabolic analyzer. FIG. 10 shows increased oxidative metabolism in AMPKγ2-transduced T cells. This was repeated in FIG. 19, which again showed an increase in oxidative metabolism of primary human T cells following transduction with AMPKγ2, a further increase in spare respiratory capacity following stimulation through the T cell receptor, and importantly an increase in extracellular acidification rates, indicative of an increase in glycolysis, in AMPKγ2-transduced that were stimulated prior to analysis. Consistent with an increase in glycolysis, AMPKγ2-transduced also had increased signaling in the mammalian target of rapamycin (mTOR) pathway, as noted in FIG. 15A-B.

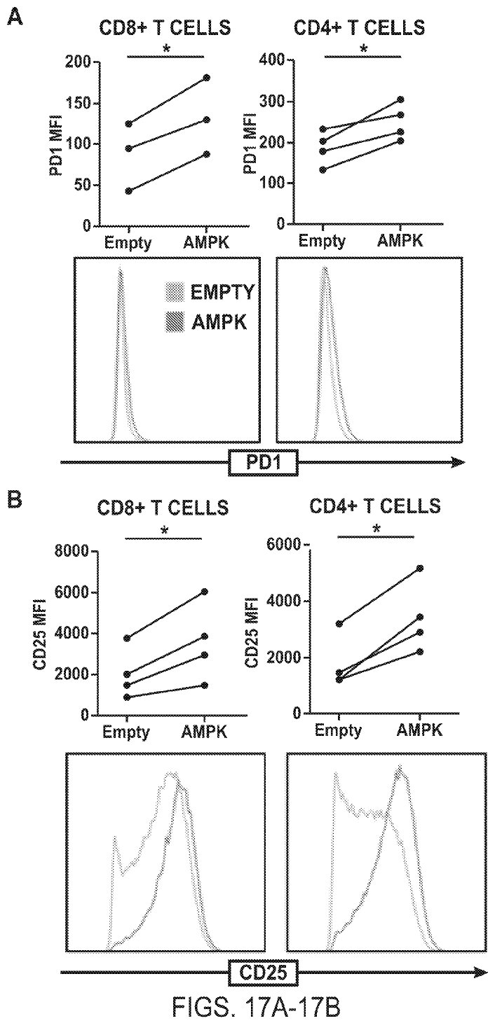

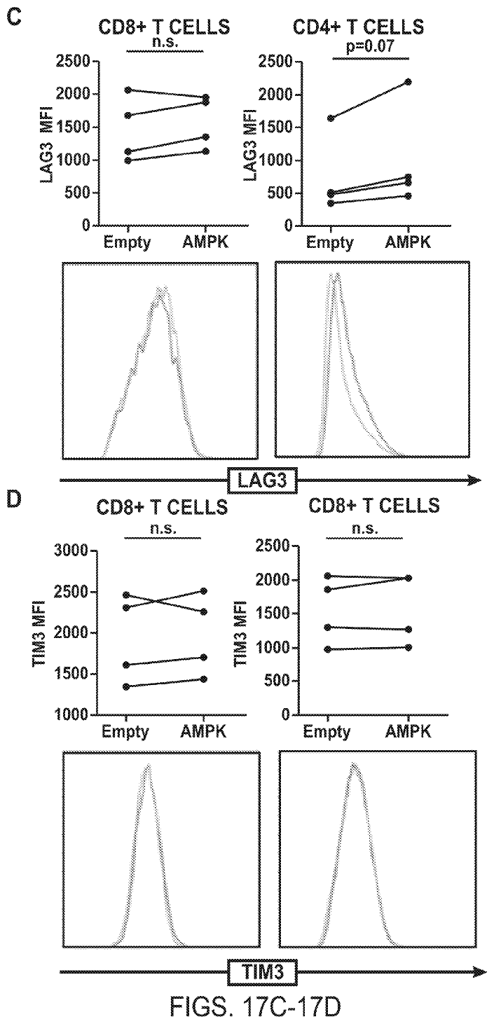

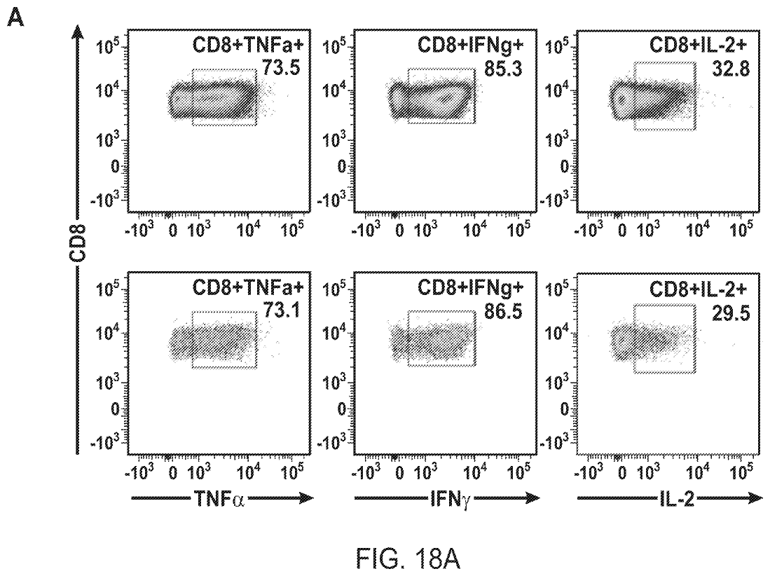

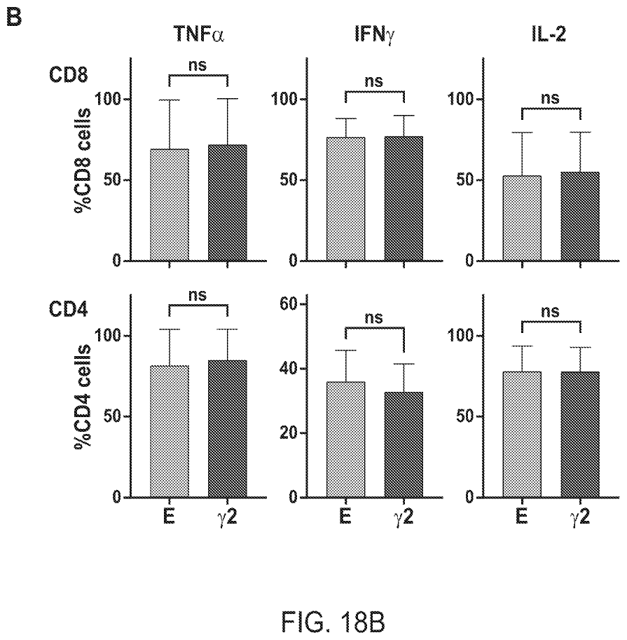

It was also important to establish whether AMPKγ2-transduced T cells were becoming exhausted. To test this, transduced cells were measured on day 10 for the activation markers PD-1 and CD25 as well as the exhaustion markers Tim3 and Lag3 (FIG. 17A-D). AMPKγ2-transduced cells had increased expression of both PD-1 and CD25, the latter of which could help explain their increased growth while being cultured in IL-2 containing media. However, neither of the exhaustion markers was upregulated on AMPKγ2-transduced cells versus controls, suggesting that AMPKγ2 transduction does not increase exhaustion. in cultured T cells. These findings were further supported by ability of AMPKγ2-transduced cells to produce equivalent levels of the pro-inflammatory cytokines interferon gamma, tumor necrosis factor alpha, and IL-2 at a similar time in culture (FIG. 18A-B).

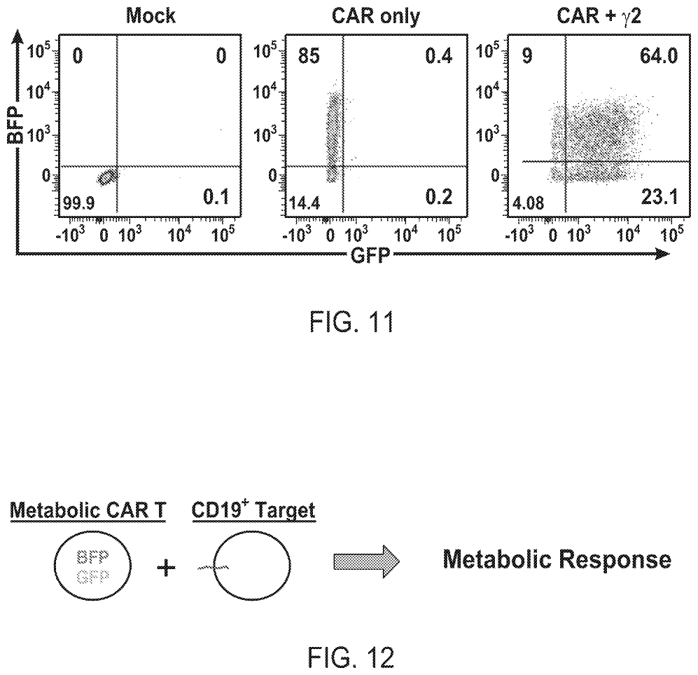

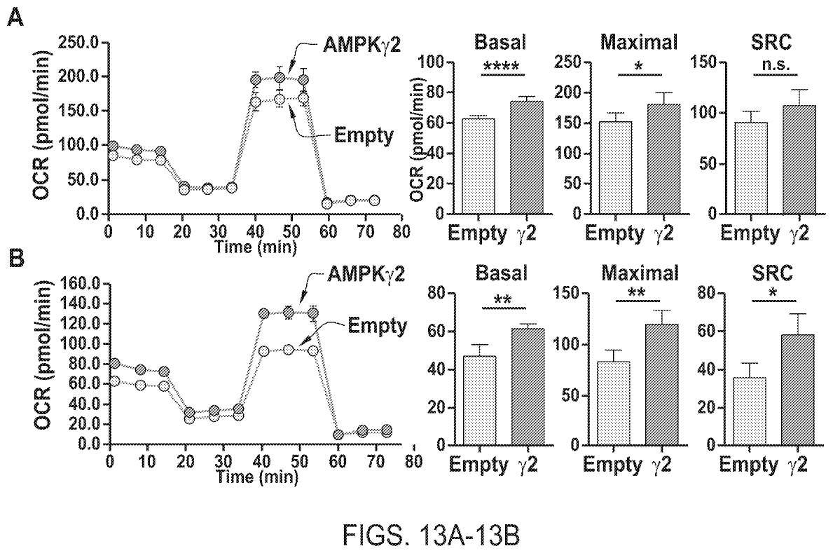

Finally, it was determined whether T cells could be co-transduced with both the AMPKγ2 construct and a chimeric antigen receptor. A Jurkat T cell line was used for the initial experiments. Mock-transduced T cells were used as a control and then either CAR-transduced T cells or T cells doubly transduced with the CAR protein and the AMPKγ2 construct were tested. Flow cytometry analysis shows successful co-transduction of CAR and AMPKγ2 (FIG. 11). Having shown co-transduction was possible, whether stimulation through the chimeric antigen receptor (CAR) can produce enhanced metabolic changes was then tested. For these experiments, dual Jurkat T cells were co-cultured with CD19-expressing target cells (targeted by the CAR) and their metabolic profiles were analyzed 24 hours later (FIG. 13). Seahorse analysis shows the increase in spare respiratory capacity, indicating increased reserves of oxidative metabolism in these cells.

1. A method of making a T cell comprising increasing a level of an AMPKγ2 polypeptide in the T cell as compared to a control, wherein the AMPKγ2 polypeptide comprises SEQ ID NO:1, and wherein the T cell having the increased level of the AMPKγ2 polypeptide has an increased oxidative metabolism as compared to the control.

2. The method of claim 1, wherein the AMPKγ2 polypeptide is at least 90% identical to SEQ ID NO:1.

3. The method of claim 1, wherein the method comprises introducing a polynucleotide sequence encoding the AMPKγ2 polypeptide into the T cell.

4. The method of claim 3, wherein the polynucleotide sequence is at least 90% identical to SEQ ID NO: 2.

5. The method of claim 3, wherein a lentiviral vector comprises the polynucleotide sequence encoding the AMPKγ2 polypeptide.

6. The method of claim 1, further comprising culturing the T cell in the presence of IL-2.

7. The method of claim 6, wherein the T cell is cultured for about 2 to 12 days.

8. The method of claim 1, wherein the T cell having the increased level of the AMPKγ2 polypeptide has one or more characteristics selected from the group consisting of:
a. is less differentiated as compared to the control,
b. has an increased level of extracellular acidification rate as compared to the control,
c. has an increased level of proliferation as compared to the control,
d. has an increased in vivo survival time as compared to the control, and
e. has increased effector function as compared to the control.

9. The method of claim 1, wherein the T cell is a CD4⁺ T cell.

10. The method of claim 1, wherein the T cell is a CD8⁺ T cell.

11. The method of claim 1, wherein the T cell is a CAR T cell, an effector T cell, an effector memory T cell, a T_{EMRA}, a central memory T cell, an effector T regulatory cell (Treg), an effector memory Treg, or a T memory stem cell (T_{SCM}).

12. The method of claim 11, wherein the T cell is an effector Treg or an effector memory Treg and has an increased suppressor function as compared to the control.

13. The method of claim 1, wherein the T cell is first obtained from a tumor, and then the level of an AMPKγ2 polypeptide is increased in the T cell as compared to the control.

14. A method of increasing an immune response in a subject comprising administering to the subject a therapeutically effective amount of a T cell having an increased level of an AMPKγ2 polypeptide as compared to a control, wherein the AMPKγ2 polypeptide comprises SEQ ID NO:1.

15. The method of claim 14, wherein the AMPKγ2 polypeptide comprises a sequence at least 95% identical to SEQ ID NO: 1.

16. The method of claim 14, wherein the T cell comprises a vector and the vector comprises a polynucleotide sequence encoding the AMPKγ2 polypeptide.

17. The method of claim 16, wherein the polynucleotide sequence is at least 90% identical to SEQ ID NO: 2.

18. The method of claim 16, wherein the vector is a lentiviral vector.

19. The method of claim 14, further comprising culturing the T cell in the presence of IL-2 prior to administration.

20. The method of claim 19, wherein the T cell is cultured for about 2 to 12 days.

21. The method of claim 14, wherein
the T cell having the increased level of the AMPKγ2 polypeptide has one or more characteristics selected from the group consisting of:
a. is less differentiated as compared to the control,
b. has an increased level of extracellular acidification rate as compared to the control,
c. has an increased level of proliferation as compared to the control,
d. has an increased in vivo survival time as compared to the control, and
e. has increased effector function as compared to the control.

22. The method of claim 14, wherein the T cell is a CD4⁺ T cell.

23. The method of claim 14, wherein the T cell is a CD8⁺ T cell.

24. The method of claim 14, wherein the T cell is a CAR T cell, an effector T cell, an effector memory T cell, a T_{EMRA}, a central memory T cell, an effector T regulatory cell (Treg), an effector memory Treg, or a T memory stem cell (T_{SCM}).

25. The method of claim 14, wherein the T cell is first obtained from a tumor, and then the level of an AMPKγ2 polypeptide is increased in the T cell as compared to the control.

26. A method of making a population of T cells comprising increasing a level of a AMPKγ2 polypeptide in the T cells as compared to a control T cell population, wherein the AMPKγ2 polypeptide comprises SEQ ID NO:1, and wherein the population of T cells has an increased amount of CD4⁺ T cells as compared to the control T cell population.


SEQUENCE LISTING (10 sequences)

SEQ ID NO: 1 - protein, 525 aa, Artificial Sequence
  Synthetic construct
MPLLDGDLEGSGKHSSRKVDSPFGPGSPSKGFFSRGPQPRPSSPMSAPVRPKTSPGSPKTVFPFSYQESPPRSPRRMSFSGIFRSSSKESSPNSNPATSPGGIRFFSRSRKTSGLSSSPSTPTQVTKQHTFPLESYKHEPERLENRIYASSSPPDTGQRFCPSSFQSPTRPPLASPTHYAPSKAAALAAALGPAEAGMLEKLEFEDEAVEDSESGVYMRFMRSHKCYDIVPTSSKLVVFDTTLQVKKAFFALVANGVRAAPLWESKKQSFVGMLTITDFINILHRYYKSPMVQIYELEEHKIETWRELYLQETFKPLVNISPDASLFDAVYSLIKNKIHRLPVIDPISGNALYILTHKRILKFLQLFMSDMPKPAFMKQNLDELGIGTYHNIAFIHPDTPIIKALNIFVERRISALPVVDESGKVVDIYSKFDVINLAAEKTYNNLDITVTQALQHRSQYFEGVVKCNKLEILETIVDRIVRAEVHRLVVVNEADSIVGIISLSDILQALILTPAGAKQKETETE

SEQ ID NO: 2 - DNA, 1581 bp, Artificial Sequence
  Synthetic construct
atgccgctcctggacggagacctggagggttccggaaagcattcctctcgaaaggtggacagccccttcggcccgggcagcccctccaaagggttcttctccagaggcccccagccccggccctccagccccatgtctgcacctgtgaggcccaagaccagccccggctctcccaaaaccgtgttcccgttctcctaccaggagtccccgccacgctcccctcgacgcatgagcttcagtgggatcttccgctcctcctccaaagagtcttcccccaactccaaccctgctacctcgcccgggggcatcaggtttttctcccgctccagaaaaacctccggcctctcctcctctccgtcaacacccacccaagtgaccaagcagcacacgtttcccctggaatcctataagcacgagcctgaacggttagagaatcgcatctatgcctcgtcttcccccccggacacagggcagaggttctgcccgtcttccttccagagcccgaccaggcctccactggcatcaccgacacactatgctccctccaaagccgcggcgctggcggcggccctgggacccgcggaagccggcatgctggagaagctggagttcgaggacgaagcagtagaagactcagaaagtggtgtttacatgcgattcatgaggtcacacaagtgttatgacatcgttccaaccagttcaaagcttgttgtctttgatactacattacaagttaaaaaggccttctttgctttggtagccaacggtgtccgagcagcgccactgtgggagagtaaaaaacaaagttttgtaggaatgctaacaattacagatttcataaatatactacatagatactataaatcacctatggtacagatttatgaattagaggaacataaaattgaaacatggagggagctttatttacaagaaacatttaagcctttagtgaatatatctccagatgcaagcctcttcgatgctgtatactccttgatcaaaaataaaatccacagattgcccgttattgaccctatcagtgggaatgcactttatatacttacccacaaaagaatcctcaagttcctccagctttttatgtctgatatgccaaagcctgccttcatgaagcagaacctggatgagcttggaataggaacgtaccacaacattgccttcatacatccagacactcccatcatcaaagccttgaacatatttgtggaaagacgaatatcagctctgcctgttgtggatgagtcaggaaaagttgtagatatttattccaaatttgatgtaattaatcttgctgctgagaaaacatacaataacctagatatcacggtgacccaggcccttcagcaccgttcacagtattttgaaggtgttgtgaagtgcaataagctggaaatactggagaccatcgtggacagaatagtaagagctgaggtccatcggctggtggtggtaaatgaagcagatagtattgtgggtattatttccctgtcggacattctgcaagccctgatcctcacaccagcaggtgccaaacaaaaggagacagaaacggaggctagc

SEQ ID NO: 3 - protein, 569 aa, Homo sapiens
MGSAVMDTKKKKDVSSPGGSGGKKNASQKRRSLRVHIPDLSSFAMPLLDGDLEGSGKHSSRKVDSPFGPGSPSKGFFSRGPQPRPSSPMSAPVRPKTSPGSPKTVFPFSYQESPPRSPRRMSFSGIFRSSSKESSPNSNPATSPGGIRFFSRSRKTSGLSSSPSTPTQVTKQHTFPLESYKHEPERLENRIYASSSPPDTGQRFCPSSFQSPTRPPLASPTHYAPSKAAALAAALGPAEAGMLEKLEFEDEAVEDSESGVYMRFMRSHKCYDIVPTSSKLVVFDTTLQVKKAFFALVANGVRAAPLWESKKQSFVGMLTITDFINILHRYYKSPMVQIYELEEHKIETWRELYLQETFKPLVNISPDASLFDAVYSLIKNKIHRLPVIDPISGNALYILTHKRILKFLQLFMSDMPKPAFMKQNLDELGIGTYHNIAFIHPDTPIIKALNIFVERRISALPVVDESGKVVDIYSKFDVINLAAEKTYNNLDITVTQALQHRSQYFEGVVKCNKLEILETIVDRIVRAEVHRLVVVNEADSIVGIISLSDILQALILTPAGAKQKETETE

SEQ ID NO: 4 - DNA, 1575 bp, Homo sapiens
atgccgctcctggacggagacctggagggttccggaaagcattcctctcgaaaggtggacagccccttcggcccgggcagcccctccaaagggttcttctccagaggcccccagccccggccctccagccccatgtctgcacctgtgaggcccaagaccagccccggctctcccaaaaccgtgttcccgttctcctaccaggagtccccgccacgctcccctcgacgcatgagcttcagtgggatcttccgctcctcctccaaagagtcttcccccaactccaaccctgctacctcgcccgggggcatcaggtttttctcccgctccagaaaaacctccggcctctcctcctctccgtcaacacccacccaagtgaccaagcagcacacgtttcccctggaatcctataagcacgagcctgaacggttagagaatcgcatctatgcctcgtcttcccccccagacacagggcagaggttctgcccgtcttccttccagagcccgaccaggcctccactggcatcaccgacacactatgctccctccaaagccgcggcgctggcggcggccctgggacccgcggaagccggcatgctggagaagctggagttcgaggacgaagcagtagaagactcagaaagtggtgtttacatgcgattcatgaggtcacacaagtgttatgacatcgttccaaccagttcaaagcttgttgtctttgatactacattacaagttaaaaaggccttctttgctttggtagccaacggtgtcagagcagcgccactgtgggagagtaaaaaacaaagttttgtaggaatgctaacaattacagatttcataaatatactacatagatactataaatcacctatggtacagatttatgaattagaggaacataaaattgaaacatggagggagctttatttacaagaaacatttaagcctttagtgaatatatctccagatgcaagcctcttcgatgctgtatactccttgatcaaaaataaaatccacagattgcccgttattgaccctatcagtgggaatgcactttatatacttacccacaaaagaatcctcaagttcctccagctttttatgtctgatatgccaaagcctgccttcatgaagcagaacctggatgagcttggaataggaacgtaccacaacattgccttcatacatccagacactcccatcatcaaagccttgaacatatttgtggaaagacgaatatcagctctgcctgttgtggatgagtcaggaaaagttgtagatatttattccaaatttgatgtaattaatcttgctgctgagaaaacatacaataacctagatatcacggtgacccaggcccttcagcaccgttcacagtattttgaaggtgttgtgaagtgcaataagctggaaatactggagaccatcgtggacagaatagtaagagctgaggtccatcggctggtggtggtaaatgaagcagatagtattgtgggtattatttccctgtcggacattctgcaagccctgatcctcacaccagcaggtgccaaacaaaaggagacagaaacggag

SEQ ID NO: 5 - DNA, 54 bp, Artificial Sequence
  Synthetic construct
gagggcagaggaagtcttctaacatgcggtgacgtggaggagaatcccggccct

SEQ ID NO: 6 - DNA, 718 bp, Artificial Sequence
  Synthetic construct
gagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaccggt

SEQ ID NO: 7 - DNA, 2633 bp, Artificial Sequence
  Synthetic construct
gctgtgaccggcgcctacgctagcatgccgctcctggacggagacctggagggttccggaaagcattcctctcgaaaggtggacagccccttcggcccgggcagcccctccaaagggttcttctccagaggcccccagccccggccctccagccccatgtctgcacctgtgaggcccaagaccagccccggctctcccaaaaccgtgttcccgttctcctaccaggagtccccgccacgctcccctcgacgcatgagcttcagtgggatcttccgctcctcctccaaagagtcttcccccaactccaaccctgctacctcgcccgggggcatcaggtttttctcccgctccagaaaaacctccggcctctcctcctctccgtcaacacccacccaagtgaccaagcagcacacgtttcccctggaatcctataagcacgagcctgaacggttagagaatcgcatctatgcctcgtcttcccccccagacacagggcagaggttctgcccgtcttccttccagagcccgaccaggcctccactggcatcaccgacacactatgctccctccaaagccgcggcgctggcggcggccctgggacccgcggaagccggcatgctggagaagctggagttcgaggacgaagcagtagaagactcagaaagtggtgtttacatgcgattcatgaggtcacacaagtgttatgacatcgttccaaccagttcaaagcttgttgtctttgatactacattacaagttaaaaaggccttctttgctttggtagccaacggtgtcagagcagcgccactgtgggagagtaaaaaacaaagttttgtaggaatgctaacaattacagatttcataaatatactacatagatactataaatcacctatggtacagatttatgaattagaggaacataaaattgaaacatggagggagctttatttacaagaaacatttaagcctttagtgaatatatctccagatgcaagcctcttcgatgctgtatactccttgatcaaaaataaaatccacagattgcccgttattgaccctatcagtgggaatgcactttatatacttacccacaaaagaatcctcaagttcctccagctttttatgtctgatatgccaaagcctgccttcatgaagcagaacctggatgagcttggaataggaacgtaccacaacattgccttcatacatccagacactcccatcatcaaagccttgaacatatttgtggaaagacgaatatcagctctgcctgttgtggatgagtcaggaaaagttgtagatatttattccaaatttgatgtaattaatcttgctgctgagaaaacatacaataacctagatatcacggtgacccaggcccttcagcaccgttcacagtattttgaaggtgttgtgaagtgcaataagctggaaatactggagaccatcgtggacagaatagtaagagctgaggtccatcggctggtggtggtaaatgaagcagatagtattgtgggtattatttccctgtcggacattctgcaagccctgatcctcacaccagcaggtgccaaacaaaaggagacagaaacggaggctagcggctccggcgagggcagaggaagtcttctaacatgcggtgacgtggaggagaatcccggccctggtaccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaccggttgagaattcgtcgagggacctaataacttcgtatagcatacattatacgaagttatacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggcttc

SEQ ID NO: 8 - DNA, 550 bp, Artificial Sequence
  Synthetic construct
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagc

SEQ ID NO: 9 - DNA, 469 bp, Artificial Sequence
  Synthetic construct
cggaacaagcctgctgtgttggatggccctgtgtctgctgggagccgatcacgccgatgcctgcccctacagcaatcccagcctgtgttctggcggcggaggcagcgaactgcctacccagggcaccttcagcaacgtgtccaccaatgtgtcccccgccaagcctaccaccaccgcctgtccttactccaaccctagcctgtgcagcggaggcggaggatctcctgctcctagacctcctacccctgcccccacaatcgccagccagcctctgtctctgaggcccgaggcttgtagacctgctgctggcggagccgtgcacaccagaggactggatttcgcctgcgacatctacatctgggcccctctggctggaacatgtggcgttctgctgctgagcctggtcatcaccctgtactgcaaccaccggaacaggcggagagtgtgcaagtgccctagacctgttgtt

SEQ ID NO: 10 - DNA, 2671 bp, Artificial Sequence
  Synthetic construct
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcatgccgctcctggacggagacctggagggttccggaaagcattcctctcgaaaggtggacagccccttcggcccgggcagcccctccaaagggttcttctccagaggcccccagccccggccctccagccccatgtctgcacctgtgaggcccaagaccagccccggctctcccaaaaccgtgttcccgttctcctaccaggagtccccgccacgctcccctcgacgcatgagcttcagtgggatcttccgctcctcctccaaagagtcttcccccaactccaaccctgctacctcgcccgggggcatcaggtttttctcccgctccagaaaaacctccggcctctcctcctctccgtcaacacccacccaagtgaccaagcagcacacgtttcccctggaatcctataagcacgagcctgaacggttagagaatcgcatctatgcctcgtcttcccccccagacacagggcagaggttctgcccgtcttccttccagagcccgaccaggcctccactggcatcaccgacacactatgctccctccaaagccgcggcgctggcggcggccctgggacccgcggaagccggcatgctggagaagctggagttcgaggacgaagcagtagaagactcagaaagtggtgtttacatgcgattcatgaggtcacacaagtgttatgacatcgttccaaccagttcaaagcttgttgtctttgatactacattacaagttaaaaaggccttctttgctttggtagccaacggtgtcagagcagcgccactgtgggagagtaaaaaacaaagttttgtaggaatgctaacaattacagatttcataaatatactacatagatactataaatcacctatggtacagatttatgaattagaggaacataaaattgaaacatggagggagctttatttacaagaaacatttaagcctttagtgaatatatctccagatgcaagcctcttcgatgctgtatactccttgatcaaaaataaaatccacagattgcccgttattgaccctatcagtgggaatgcactttatatacttacccacaaaagaatcctcaagttcctccagctttttatgtctgatatgccaaagcctgccttcatgaagcagaacctggatgagcttggaataggaacgtaccacaacattgccttcatacatccagacactcccatcatcaaagccttgaacatatttgtggaaagacgaatatcagctctgcctgttgtggatgagtcaggaaaagttgtagatatttattccaaatttgatgtaattaatcttgctgctgagaaaacatacaataacctagatatcacggtgacccaggcccttcagcaccgttcacagtattttgaaggtgttgtgaagtgcaataagctggaaatactggagaccatcgtggacagaatagtaagagctgaggtccatcggctggtggtggtaaatgaagcagatagtattgtgggtattatttccctgtcggacattctgcaagccctgatcctcacaccagcaggtgccaaacaaaaggagacagaaacggaggctagcggctccggcgagggcagaggaagtcttctaacatgcggtgacgtggaggagaatcccggccctggtaccggaacaagcctgctgtgttggatggccctgtgtctgctgggagccgatcacgccgatgcctgcccctacagcaatcccagcctgtgttctggcggcggaggcagcgaactgcctacccagggcaccttcagcaacgtgtccaccaatgtgtcccccgccaagcctaccaccaccgcctgtccttactccaaccctagcctgtgcagcggaggcggaggatctcctgctcctagacctcctacccctgcccccacaatcgccagccagcctctgtctctgaggcccgaggcttgtagacctgctgctggcggagccgtgcacaccagaggactggatttcgcctgcgacatctacatctgggcccctctggctggaacatgtggcgttctgctgctgagcctggtcatcaccctgtactgcaaccaccggaacaggcggagagtgtgcaagtgccctagacctgttgtttaa